(19) United States
(12) Patent Application Publication
(10) Pub. No.: US 2021/0139579 A1
(43) Pub. Date: May 13, 2021

(54) MULTISPECIFIC ANTIBODY PRODUCT THAT BINDS TO DIFFERENT ROR1 EPITOPES

(71) Applicant: NBE-Therapeutics AG, Basel (CH)

(72) Inventors: Ulf GRAWUNDER, Hersberg (CH); Roger BEERLI, Aldikon bei Regensdorf (CH); Lorenz WALDMEIER, Basel (CH)

(21) Appl. No.: 16/629,910

(22) PCT Filed: Jul. 20, 2018
(86) PCT No.: PCT/EP2018/069798

§ 371 Date: Jan. 9, 2020

(30) Foreign Application Priority Data
Jul. 20, 2017 (EP) 17182420.4

(51) Int. Cl. C07K 16/28; A61K 47/68

(52) U.S. Cl. CPC C07K 16/2803; A61K 47/6803; C07K 2317/32; C07K 2317/73; C07K 2318/20; C07K 2317/31

(57) ABSTRACT

The present invention relates to a multi-specific product comprising a first entity comprising an antigen-binding domain that binds to the same ROR1 epitope as and/or competes for ROR1 binding with an antibody comprising a heavy chain variable region sequence shown in SEQ ID NO. 2 and a light chain variable region sequence shown in SEQ ID NO. 3; and a second entity comprising an antigen-binding domain that binds to a different target or ROR1 epitope than, and/or does not compete for binding with the antibody comprising a heavy chain variable region sequence shown in SEQ ID NO. 2 and a light chain variable region sequence shown in SEQ ID NO. 3.

### RELATED APPLICATIONS

This application is a 35 U.S.C. § 371 filing of International Patent Application No. PCT/EP2018/069798, filed Jul. 20, 2018, which claims priority to European Patent Application No. 17182420.4, filed Jul. 20, 2017, the entire disclosures of which are hereby incorporated herein by reference.

### FIELD OF THE INVENTION

The present invention relates to a multispecific antibody product that binds to a first ROR1 epitope and to at least one other epitope of ROR1, and conjugates thereof, as well as to uses thereof.

### BACKGROUND OF THE INVENTION

Cancer is one of the leading causes of death. It is a class of diseases caused by malignant transformation of healthy cells, resulting from genetic alterations, like chromosomal translocations, mutations in tumor suppressor genes, transcription factors or growth-factor receptors, leading to the immortalization of the cells. If the immortalization is combined with excessive proliferation, the immortalized cells generate tumors, with or without metastasis (in case of solid tumors), or leukemias and lymphomas (cancers of the blood). Defective apoptosis, or programmed cell death, can further contribute to malignant transformation of cells leading to cancer.

A family of membrane associated receptor tyrosine kinases, consisting of the receptor tyrosine kinase orphan receptors-1 and -2 (ROR1 and ROR2) have been described as specifically associated with particular cancers (Rebagay et al. (2012) Front Oncol. 2(34):1-8; doi 10.3389/onc.2012.00034), while being largely absent in expression on healthy tissue with, a few exceptions e.g. in case of ROR1 (Balakrishnan et al. (2016) Clin Cancer Res. doi: 10.1158/1078-0432). Whether or not ROR expression is functionally associated with tumorigenesis remains unclear. However, due to the very tumor-selective expression of ROR family members, they represent relevant targets for targeted cancer therapies.

Receptor tyrosine kinase orphan receptors-1 and -2, ROR1 and ROR2, are the only two family members defining a new receptor tyrosine kinase family, based on the overall structural design and some functional similarities. Both ROR1 and ROR2 proteins are type I-single pass trans-membrane receptors with an extracellular domain (ECD) consisting of an immunoglobulin domain, a cysteine rich frizzled domain and a Kringle domain. These three extracellular domains are followed by a trans-membrane domain connecting the ECD to an intracellular portion of the protein comprising kinase domains (Rebagay et al. (2012) Frontiers Oncol. 2(34):1-8; doi 10.3389/onc.2012.00034).

The human ROR1 and ROR2 proteins are 58% homologous between each other, but each of the ROR proteins is highly conserved between species. This represents a challenge for the development of human ROR1 specific monoclonal antibodies and very few antibodies are known.

Further, it appears that anti-ROR1 antibodies, and antibody drug conjugates (ADCs) that encompass anti-ROR1 antibodies, show only limited efficacy, in particular on cell lines and tumors with low expression levels of ROR1.

It is hence one object of the present invention to provide antibody-based products that target ROR1 and demonstrate a better efficacy, in particular on cell lines and tumors with low expression levels of ROR1.

It is another object of the present invention to provide antibody drug conjugates (ADCs) that target ROR1 and demonstrate a better efficacy, in particular on cell lines with low expression levels.

These and further objects are met with methods and means according to the independent claims of the present invention. The dependent claims are related to specific embodiments.

### SUMMARY OF THE INVENTION

The present invention relates to a multi-specific product that binds to a first ROR1 epitope and to at least one other epitope on ROR1, and conjugates thereof, as well as the uses thereof. The invention and general advantages of its features will be discussed in detail below.

### DETAILED DESCRIPTION OF THE FIGURES

FIG. 1 shows the amino acid sequences of variable immunoglobulin heavy and light chains of novel rabbit anti-human ROR1 (hROR1) mAbs, as indicated. The amino acid sequence alignment of the rabbit variable domains (V_{κ}, V_{λ}, and V_{H}) is shown with framework regions (FR) and complementarity determining regions (CDR) using Kabat numbering. Shown in the figure are the heavy chain variable domain sequences and the light chain variable domain sequences of 13 antibodies designated XBR1-402, ERR1-301, ERR1-306, ERR1-316, ERR1-324, ERR1-403, ERR1-409, ERR1-TOP4, ERR1-TOP15, ERR1-TOP22, ERR1-TOP40, ERR1-TOP43, and ERR1-TOP54. As indicated in the figure clones XBR1-402, ERR1-301, ERR1-306, ERR1-316, ERR1-403, ERR1-409, ERR1-TOP4, ERR1-TOP15, ERR1-TOP22, ERR1-TOP43, and ERR1-TOP54 are variable domains of immunoglobulin λ light chains, while antibodies ERR1-324 and ERR1-TOP40 are variable domains of immunoglobulin κ light chains.

FIG. 2 shows the binding activity of chimeric rabbit/human Fabs to human ROR1 (hROR1) and mouse ROR1 (mROR1) expressed as fusion proteins of the extracellular domain (ECD) of hROR1 and mROR1 to the human Fc domain of a human IgG1 antibody. The binding of each chimeric rabbit/human Fab to hROR1 and mROR1 fused with human IgG1 Fc (hFc-hROR1 and hFc-mROR1) was analyzed by ELISA. hFc-ROR1 or hFc-mROR1 were captured by anti-human IgG1 Fc antibody immobilized on plate and then incubated with hROR1 specific Fabs comprising a His-tag via detection with mouse anti-His tag. Specificity of the Fabs was confirmed by using fusion proteins of the extracellular domain (ECD) of hROR2 with the human Fc domain of a human IgG1 antibody (hFc-hROR2) and with bovine serum albumin (BSA) as control.

FIG. 3 shows binding activity of chimeric rabbit/human Fabs to native human ROR1 protein expressed on the cell surface of murine preB cell line 63-12 (see Example 1). The binding of each chimeric rabbit/human Fab to the ectopically expressed human ROR1 on mouse pre-B cell (63-12) surface was analyzed by flow cytometry. ERR2-TOP35 is a mAb against hROR2 that served as an isotype-matched control.

FIG. 4 shows epitope mapping studies for chimeric rabbit/human Fabs on six different immobilized IgG1-Fc fusion proteins that comprise different parts of the extracellular domain of human ROR1: hFc-hROR1-Ig (comprising the Immunoglobulin-domain of hROR1), hFc-hROR1-Fr (comprising the Frizzled domain of hROR1), hFc-hROR1-Kr (comprising the Kringle domain of hROR1), hFc-hROR1-Ig-Fr (comprising the Immunoglobulin and Frizzled domains of hROR1), hFc-hROR1-Fr-Ki (comprising the Frizzled and Kringle domains of hROR1) and hFc-hROR1 (comprising the entire extracellular domain (ECD) of hROR1).

FIG. 5 shows epitope binding studies performed by surface plasmon resonance. Shown are SPR sensorgrams obtained for the binding of different Fabs to hFc-hROR1 captured by anti-human Fcγ antibody immobilized on a CMS chip. Fabs were injected in different orders to identify independent and overlapping epitopes. Resonance unit (RU, y axis) increases that exceeded the values found for previously injected Fabs indicated independent epitopes because they allow simultaneous binding. For example, the increase found for the binding of Fab R11 exceeded the values found for XBR1-402 alone, indicating that Fab R11 and XBR1-402 can bind simultaneously to human ROR1. By contrast, the epitope of Fab XBR1-402 overlaps with the epitopes of ERR1-301, ERR1-403 and R12 (left graph); the epitope of Fab ERR1-TOP43 overlaps with the epitope of ERR1-306, XBR1-402 and ERR1-TOP40. The x-axis depicts the time in seconds (s).

FIGS. 6A-6B show sensorgrams of affinity measurements of anti-hROR1 specific Fabs to hROR1 ECD by surface plasmon resonance (SPR). (FIG. 6A) Shown are Biacore X100 sensorgrams obtained for the binding of each Fab to hFc-hROR1 captured by anti-human Fcγ antibody immobilized on CMS chip after instantaneous background depletion. Fabs were injected at five different concentrations with the highest concentration indicated in FIG. 6(B), one of the five concentrations was tested in duplicates. (FIG. 6B) Monospecific affinities of each Fab are shown in the table. The equilibrium dissociation constant (K_{d}) was calculated from k_{off}/kₒₙ (kₒₙ, association rate constant; k_{off}, dissociation rate constant).

FIGS. 7A-7B shows the binding analyzed by ELISA of selected hROR1 specific rabbit-human-Fc chimeric antibodies of selected clones ERR1-301, XBR1-402, ERR1-306, ERR1-324, ERR1-403 and ERR1-Top43 to recombinant, purified hROR1 (FIG. 7A) and to recombinant, purified hROR2 as a negative control (FIG. 7B).

FIG. 8 shows FACS-based cell staining of hROR1 on various human cancer cell lines with anti-human ROR1 antibody 2A2 as described in Example 9. Cell lines analyzed include 697 (human acute lymphocytic leukemia, ALL), human triple-negative breast cancer cell lines MDA-MB-468 and HS-578T, human lung cancer cell line A549, human colon cancer cell line HT-29, as well as human breast cancer cell line T47D. Except for the T47D human breast cancer cell line, all of the evaluated cells are positive for hROR1 expression.

FIGS. 9A-9B shows schematically how site-specifically conjugated ADCs disclosed in this invention have been generated. (FIG. 9A) schematically shows the mechanism of sortase-enzyme mediated antibody conjugation (SMAC-technology) as disclosed in WO2014140317. In order to generate site-specifically conjugated ADCs, recombinant antibodies need to be expressed with the C-terminal pentapeptide motif LPXTG, which serve as recognition sites for the sortase A enzyme from Staphylococcus aureus (SrtA). When a glycine modified toxin substrate is incubated with pentapeptide motif LPXTG containing antibody and sortase A enzyme, the sortase A enzyme catalyzes a transpeptidation reaction by which the glycine-modified toxin replaces the C-terminal glycine of the LPXTG motif and is covalently coupled to the threonine of the remaining LPXT sequence. This way C-terminally toxin-conjugated ADCs can be generated with high efficiency. (FIG. 9B) shows the structure of the preferred toxin, a PNU-159682 derivative comprising an ethylene-diamino (EDA) linker connecting a 5× glycine stretch to the carbonyl group at C13 of the anthracycline structure, as disclosed in WO2016102697.

FIGS. 10A-10E show in vitro cell killing assays performed on human ALL cancer cell line 697 with individual antibody-based ADCs and mixtures thereof comprising ERR1-324-based ADCs as per Example 10. For the same total dose of ADC, the mixtures of selected ADCs of the invention provide synergistic killing of 697 cancer cells that is superior to the individual ADCs.

FIG. 11 shows evaluation of in vitro cell killing performed on ROR1 positive human ALL cancer cell line 697 with individual scFv-Fc-G5-PNU-toxin conjugates based on anti-ROR1 antibody XBR1-402 and anti-ROR1 antibody ERR1-324 and also mixtures of these two scFv-Fcs at the combined same concentration as used for the single scFv-Fcs, as well as a bi-epitope-reactive scFv-Fc-based ADCs (BETR-ADC™), in which the two binding domains are combined in a single, bi-specific antibody molecule, as indicated. For the same total dose of ADC, the mixtures of selected ADCs and the bi-epitope-reactive scFv-Fc-based ADC (BETR-ADC™) of the invention provide improved cell killing activity of 697 cancer cells that is superior to the cell killing activity on ROR1 positive 697 cells of individual ADCs at equivalent total concentration.

FIG. 12 shows evaluation of in vitro cell killing performed on human ALL cancer cell line 697 with individual DVD-Ig-based bi-epitope-reactive anti-ROR1 ADCs (BETR-ADC™) based on anti-ROR1 antibody XBR1-402 and anti-ROR1 antibody ERR1-324 and mixtures of ADCs based on anti-ROR1 antibody XBR1-402 and anti-ROR1 antibody ERR1-324. For the same total dose of ADC, the bi-epitope-reactive DVD-Ig-based anti-ROR1 ADC of the invention show cell killing activity of 697 cancer cells that is superior to the cell killing activity on ROR1 positive 697 cells of individual ADCs at equivalent total concentration. Trastuzumab-PNU ADC (Tras-G5-PNU) was used as an isotype-matched control ADC.

FIGS. 13A-13B show evaluation of in vitro cell killing performed on ROR1-positive human cancer cell lines (colon cancer cell line HT-29, triple-negative breast cancer cell lines MDA-MB-468 and HS-578T, lung cancer cell line A549,) with individual anti-ROR1 specific antibody-based ADCs (FIG. 13A) and mixtures thereof (FIG. 13B), as indicated, and comprising anti-ROR1 ERR1-324-based ADCs as per Example 13. For the same total dose of ADC, the mixtures of selected ADCs of the invention provide synergistic target cell killing on ROR1-positive cell lines that is superior to the target cell killing with individual ADCs. CD30-specific PNU-ADC based on brentuximab (clone Ac10) was used as an isotype-matched control ADC.

FIGS. 14A-14C show three different embodiments of the invention that have experimentally been evaluated. FIG. 14A shows the use of mixtures of two ADCs comprising a monospecific anti-ROR1 antibody each that bind to different epitopes of a ROR1 target molecule. The two monospecific antibodies target two different epitopes of ROR1, one of which competes for binding to ROR1 with an antibody having SEQ ID NOs 2 and 3. FIG. 14B shows the use of a bi-epitope-reactive ADC (BETR-ADC′) comprising a bi-epitope-reactive antibody in the scFv-Fc format. The two target binding domains of the two scFv subunits bind to two different epitopes of ROR1, one of which competes for binding to ROR1 with an antibody having SEQ ID NOs 2 and 3. FIG. 14C shows the use of a bi-epitope-reactive ADC (BETR-ADC′) comprising a bi-epitope-reactive antibody in the DVD-Ig format. The two target binding domains bind to two different epitopes of ROR1, one of which competes for binding to ROR1 with an antibody having SEQ ID NOs 2 and 3.

FIG. 15 schematically shows the structures of the three bi-epitope-reactive antibody formats scFv-Fc, DVD-Ig and bispecific scFv-Ig. Note that in a knob-in-hole embodiment, the two Fc domains can be structurally modified, as, e.g., shown in Shatz et al., mAbs 5:6, 872-881(2013). Note also the respectively modified sequences in the sequence listing.

FIGS. 16A-16B show how recognition of two independent epitopes by bi-epitope-reactive ADC (BETR-ADC™) can not only induce the formation of target-homodimers (FIG. 16A), but lead to extensive clustering of the target protein (FIG. 16B). This in turn may enhance internalization of said product, a more efficient transport to intracellular lysosomes, and, in the case of an ADC, ultimately facilitate degradation-dependent release of the payload, thus leading to an increase of potency for killing of target expressing cells.

FIG. 17 shows evaluation of in vitro cell killing performed, on murine cancer cell line EMT-6 engineered to overexpress ROR1, with an scFv-IgG-based bi-epitope-reactive anti-ROR1 ADC (BETR-ADC′) based on anti-ROR1 antibody XBR1-402 and anti-ROR1 antibody ERR1-324. For the same total dose of ADC, the bi-epitope-reactive scFv-IgG-based anti-ROR1 ADC of the invention show cell killing activity on engineered EMT-6 cancer cells that is superior to the cell killing activity of individual ADCs at equivalent total concentration. Trastuzumab-PNU ADC (Tras-G3-PNU) was used as an isotype-matched control ADC.

### DETAILED DESCRIPTION OF THE INVENTION

Before the invention is described in detail, it is to be understood that this invention is not limited to the particular component parts of the devices described or process steps of the methods described as such devices and methods may vary. It is also to be understood that the terminology used herein is for purposes of describing particular embodiments only, and is not intended to be limiting. It must be noted that, as used in the specification and the appended claims, the singular forms “a”, “an”, and “the” include singular and/or plural referents unless the context clearly dictates otherwise. It is moreover to be understood that, in case parameter ranges are given which are delimited by numeric values, the ranges are deemed to include these limitation values.

It is further to be understood that embodiments disclosed herein are not meant to be understood as individual embodiments which would not relate to one another. Features discussed with one embodiment are meant to be disclosed also in connection with other embodiments shown herein. If, in one case, a specific feature is not disclosed with one embodiment, but with another, the skilled person would understand that does not necessarily mean that said feature is not meant to be disclosed with said other embodiment. The skilled person would understand that it is the gist of this application to disclose said feature also for the other embodiment, but that just for purposes of clarity and to keep the specification in a manageable volume this has not been done.

Furthermore, the content of the prior art documents referred to herein is incorporated by reference. This refers, particularly, for prior art documents that disclose standard or routine methods. In that case, the incorporation by reference has mainly the purpose to provide sufficient enabling disclosure, and avoid lengthy repetitions.

According to a first aspect of the invention, a multispecific antibody-based product comprising
(a) a first entity comprising an antigen-binding domain that
   binds to the same ROR1 epitope as and/or
   competes for ROR1 binding with
an antibody comprising a heavy chain variable region sequence shown in SEQ ID NO. 2 and a light chain variable region sequence shown in SEQ ID NO. 3; and
(b) a second entity comprising an antigen-binding domain that binds to
   to a different ROR1 epitope than, and/or
   does not compete for binding with
the antibody comprising a heavy chain variable region sequence shown in SEQ ID NO. 2 and a light chain variable region sequence shown in SEQ ID NO. 3.

As herein, the term “multispecific” means a product which has specificity for two or more different epitopes of ROR-1 antigens. In the context of the present invention, the terms “multispecific” and “multi-epitope reactive” are hence used simultaneously. In one embodiment, the multi-epitope reactive product is bi-epitope reactive.

The term “variable region”, as used herein, refers to the respective regions of the heavy and light chain of an antibody, abbreviated V_{H} and V_{L}, (sometimes also written V_{H} and V_{L}, or HCVD and LCVD), as opposed to the constant domains C_{H}1, C_{H}2 and C_{H}3 of the heavy chain and C_{L} of the light chain. The variable regions encompass the complementarity determining regions (CDRs). The term “variable domain” is used interchangeably with the term “variable region” herein.

While an antibody comprising an Fc region has specificity not only for antigen epitopes, via its VH/VL domains, but also binds, via its Fc region, to an Fc receptor. In case its VH/VL domains bind two different epitopes, such antibody will still be called “bispecific” or “bi-epitope reactive”, in case its VH/VL domains bind two different epitopes, despite the fact that its actually binds another target, namely an Fc receptor. In case its VH/VL domains bind only one epitope, such antibody will still be called “monospecific” or “mono-epitope reactive”, in case its VH/VL domains bind two different epitopes, despite the fact that its actually binds another target, namely an Fc receptor.

According to one embodiment of the respective aspect of the invention, the product is a multispecific antibody, alternative scaffold or antibody mimetic wherein
(a) the first entity is a first antigen-binding domain that
   binds to the same ROR1 epitope as and/or
   competes for ROR1 binding with
antibody ERR1-324 comprising a heavy chain variable region sequence shown in SEQ ID NO. 2 and a light chain variable region sequence shown in SEQ ID NO. 3; and
(b) the second entity is a second antigen-binding domain that
   binds to a different ROR1 epitope than, and/or
   does not compete for binding with
antibody ERR1-324 comprising a heavy chain variable region sequence shown in SEQ ID NO. 2 and a light chain variable region sequence shown in SEQ ID NO. 3.

According to another embodiment of the respective aspect of the invention, the product comprises two or more antibodies, alternative scaffolds or antibody mimetics wherein
(a) the first entity is a first antibody comprising an antigen-binding domain that
   binds to the same ROR1 epitope as and/or
   competes for ROR1 binding with
antibody ERR1-324 comprising a heavy chain variable region sequence shown in SEQ ID NO. 2 and a light chain variable region sequence shown in SEQ ID NO. 3; and
(b) the second entity is a second antibody comprising an antigen-binding domain that
   binds to a different ROR1 epitope than, and/or
   does not compete for binding with
antibody ERR1-324 comprising a heavy chain variable region sequence shown in SEQ ID NO. 2 and a light chain variable region sequence shown in SEQ ID NO. 3.

The two embodiments discussed above are shown, in principle, in FIG. 14. Both have in common that they relate to a multispecific product wherein
a first entity binds to the same ROR1 epitope as and/or competes for ROR1 binding with an antibody having SEQ ID NO. 2 (HCVD) and SEQ ID NO. 3 (LCVD) and
a second entity binds to a different ROR1 epitope than, and/or does not compete for ROR1 binding with the antibody having SEQ ID NO. 2 (HCVD) and SEQ ID NO. 3 (LCVD).

These two entities can either be on two different antibodies, or on a single multi-specific antibody molecule.

Additionally, the invention provides bi- or multispecific antibodies targeting ROR1 as well as at least one binding domain specific for another target, for instance, but not limited to targets that recruit and/or activate cells of the immune system, like T cells or NK cells. Such other binding domains may be specific for CD3, CD16, CD32, CD56, CD64 or other markers specific for T and NK cells.

SEQ ID NOs 2 and 3 belong to an anti-ROR1 antibody called ERR1-324. This antibody binds a specific epitope of ROR1, including human ROR1 (hROR1). In a preferred embodiment, the first entity comprises the CDRs of ERR1-324, as given in FIG. 1, i.e., the first entity comprises the following CDRs: QASQSVYGNNELA (V_{K} CDR1), RASILTS (V_{K} CDR2), LGGYVSQSYRAA (V_{K} CDR3), RNGMT (V_{H} CDR1), IITSSGDKYYATWAKG (V_{H} CDR2), and GTVSSDI (V_{H} CDR3).

In a preferred embodiment, the first entity comprises the variable domain sequences of ERR1-324, i.e., comprises the variable domain sequences of SEQ ID NO. 2 (HCVD) and SEQ ID NO. 3 (LCVD).

The invention shows that such multi-specific product has significant advantages over a product which only has a single epitope reactivity for the ROR1 target, like e.g. the epitope that an antibody having SEQ ID NO. 2 (HCVD) and SEQ ID NO. 3 (LCVD).

Without being bound to theory, it is conceivable that the bi-epitope reactivity may induce the formation of target-homodimers or even clusters of the target as schematically presented in FIG. 16. This may increase the induction of internalization of the said product by receptor-mediated endocytosis, and therefore a more efficient transport of such bi-epitope reactive ADCs (BETR-ADC™) to intracellular lysosomes leading to higher potency for killing of target expressing cells.

According to one embodiment, the entity comprising an antigen-binding domain is at least one selected from the group consisting of an antibody, an antibody-based binding protein, a modified antibody format retaining target binding capacity, an antibody derivative or a fragment retaining target binding capacity, an alternative scaffold and/or an antibody mimetic.

According to one other embodiment, the antibody is at least one selected from the group consisting of an an antibody, an antibody-based binding protein, a bi-epitope-reactive antibody, a modified antibody format retaining target binding capacity, an antibody derivative or a fragment retaining target binding capacity, an alternative scaffold and/or an antibody mimetic.

According to one other embodiment, the antibody is at least one selected from the group consisting of an antibody, an antibody-based binding protein, a bi-epitope-reactive antibody, a modified antibody format retaining target binding capacity, an antibody derivative or a fragment retaining target binding capacity.

The term “fully human antibody” refers to an antibody, antibody-based binding protein or antigen-binding fragment that contains sequences derived from human immunoglobulin genes, such that substantially all of the heavy and light chain CDR1 and CDR2 regions are of human origin, and substantially all of the heavy and light chain FR regions 1, 2, 3, and 4 correspond to those of a human immunoglobulin sequence either with or without a limited number of somatic mutations that may be introduced into individual heavy and light chain CDR1 and CDR2 and FR1, 2, 3, and 4 variable domain sequences.

The terms “antibody”, “antibody-based binding protein”, “modified antibody format retaining target binding capacity”, “antibody derivative or fragment retaining target binding capacity” refers to polypeptide chain(s) which exhibit a strong monovalent, bivalent or polyvalent binding to a given antigen, epitope or epitopes. Antibodies, antibody-based binding proteins and antigen-binding fragments used in the invention can be generated using any suitable technology, e.g., hybridoma technology, ribosome display, phage display, gene shuffling libraries, semi-synthetic or fully synthetic libraries or combinations thereof. Antibodies, antibody-based binding proteins and antigen-binding fragments of the invention include intact antibodies and antibody fragments or antigen-binding fragments that contain the antigen-binding portions of an intact antibody and retain the capacity to bind the cognate antigen. Unless otherwise specified herein, all peptide sequences, including all antibody and antigen-binding fragment sequences are referred to in N->C order.

An intact antibody typically comprises at least two heavy (H) chains (about 50-70 kD) and two light (L) chains (about 25 kD) inter-connected by disulfide bonds. The recognized immunoglobulin genes encoding antibody chains include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Each heavy chain of an antibody is comprised of a heavy chain variable region (VH) and a heavy chain constant region. In the case of IgG, the heavy chain constant region is comprised of three domains, CH1, CH2 and CH3. Each light chain is comprised of a light chain variable region (VL) and a light chain constant region. The light chain constant region is comprised of one domain, CL. The variable regions of the heavy and light chains contain a binding domain that interacts with an antigen. The constant regions of the antibodies may mediate the binding of the immunoglobulin to host tissues or factors, including various cells of the immune system and the first component (Clq) of the classical complement system. Monoclonal antibodies (mAbs) consist of identical antibodies molecules.

The VH and VL regions of an antibody can be further subdivided into regions of hypervariability, also termed complementarity-determining regions (CDRs), which are interspersed with the more conserved framework regions (FRs). Each VH and VL is composed of three CDRs and four FRs, arranged from amino-terminus to carboxyl-terminus in the following order: FR1, CDR1, FR2, CDR2, FR3, CDR3, FR4. The locations of CDR and FR regions and a numbering system have been defined, e.g., the IMGT system (Lefranc M P et al., 2015), or the Kabat numbering scheme.

Antibodies, antibody-based binding proteins and antigen-binding fragments of the invention also encompass single chain antibodies. The term “single chain antibody” refers to a polypeptide comprising a VH domain and a VL domain in polypeptide linkage, generally linked via a spacer peptide, and which may comprise additional domains or amino acid sequences at the amino- and/or carboxyl-termini. For example, a single-chain antibody may comprise a tether segment for linking to the encoding polynucleotide. As an example, a single chain variable region fragment (scFv) is a single-chain antibody. Compared to the VL and VH domains of the Fv fragment that are coded for by separate genes, a scFv has the two domains joined (e.g., via recombinant methods) by a synthetic linker. This enables them to be made as a single protein chain in which the VL and VH regions pair to form monovalent molecules.

Examples of antibody-based binding proteins are polypeptides in which the binding domains of the antibodies are combined with other polypeptides or polypeptide domains, e.g. alternative molecular scaffolds, Fc-regions, other functional or binding domains of other polypeptides or antibodies resulting in molecules with addition binding properties, e.g. bi- or multispecific proteins or antibodies. Such polypeptides can create an arrangement of binding or functional domains normally not found in naturally occurring antibodies or antibody fragments.

Examples of antigen-binding fragments include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an intact antibody; (v) disulfide stabilized Fvs (dsFvs) which have an interchain disulfide bond engineered between structurally conserved framework regions; (vi) a single domain antibody (dAb) which consists of a VH or VL domain (see, e.g., Ward et al., Nature 341:544-546, 1989); and (vii) an isolated complementarity determining region (CDR) as a linear or cyclic peptide.

Antigen-binding fragments of the present invention also encompass single domain antigen-binding units that have a camelid scaffold. Animals in the camelid family include camels, llamas, and alpacas. Camelids produce functional antibodies devoid of light chains. The heavy chain variable (VH) domain folds autonomously and functions independently as an antigen-binding unit. Its binding surface involves only three CDRs as compared to the six CDRs in classical antigen-binding molecules (Fabs) or single chain variable fragments (scFvs). Camelid antibodies are capable of attaining binding affinities comparable to those of conventional antibodies.

The terms “alternative scaffold” and “antibody mimetic” refer to proteins not belonging to the immunoglobulin family, and even non-proteins such as aptamers, or synthetic polymers. Some types have an antibody-like beta-sheet structure. Potential advantages of “antibody mimetics” or “alternative scaffolds” over antibodies are better solubility, higher tissue penetration, higher stability towards heat and enzymes, and comparatively low production costs.

Some antibody mimetics can be provided in large libraries, which offer specific binding candidates against every conceivable target. Just like with antibodies, target specific antibody mimetics can be developed by use of High Throughput Screening (HTS) technologies as well as with established display technologies, just like phage display, bacterial display, yeast or mammalian display. Currently developed antibody mimetics encompass, for example, ankyrin repeat proteins (called DARPins), C-type lectins, A-domain proteins of S. aureus, transferrins, lipocalins, 10th type III domains of fibronectin, Kunitz domain protease inhibitors, ubiquitin derived binders (called affilins), gamma crystallin derived binders, cysteine knots or knottins, thioredoxin A scaffold based binders, nucleic acid aptamers, artificial antibodies produced by molecular imprinting of polymers, peptide libraries from bacterial genomes, SH-3 domains, stradobodies, “A domains” of membrane receptors stabilised by disulfide bonds and Ca2+, CTLA4-based compounds, Fyn SH3, and aptamers (oligonucleic acid or peptide molecules that bind to a specific target molecules)

The anti-ROR1 antibodies, antibody-based binding proteins and antigen-binding fragments described herein can be produced by enzymatic or chemical modification of the intact antibodies, or synthesized de novo using recombinant DNA methodologies. Methods for generating these antibodies, antibody-based binding proteins and antigen-binding molecules are all well known in the art. In particular, scFv antibodies can be obtained using methods described in, e.g., Bird et al., Science 242:423-426, 1988; and Huston et al., Proc. Natl. Acad. Sci. USA 85:5879-5883, 1988. Fv antibody fragments can be generated as described in Skerra and Plückthun, Science 240:1038-41, 1988. Disulfide-stabilized Fv fragments (dsFvs) can be made using methods described in, e.g., Reiter et al., Int. J. Cancer 67:113-23, 1996. Similarly, single domain antibodies (dAbs) can be produced by a variety of methods described in, e.g., Ward et al., Nature 341:544-546, 1989; and Cai and Garen, Proc. Natl. Acad. Sci. USA 93:6280-85, 1996. Camelid single domain antibodies can be produced using methods well known in the art, e.g., Dumoulin et al., Nat. Struct. Biol. 11:500-515, 2002; Ghahroudi et al., FEBS Letters 414:521-526, 1997; and Bond et al., J. Mol. Biol. 332:643-55, 2003. Other types of antigen-binding fragments (e.g., Fab, F(ab′)2 or Fd fragments) can also be readily produced with routinely practiced immunology methods. See, e.g., Harlow & Lane, Using Antibodies, A Laboratory Manual, Cold Spring Harbor Laboratory Press, Cold Spring Harbor, N.Y., 1998.

The anti-ROR1 antibodies, antibody-based binding proteins or antigen-binding fragments of the invention can be produced by any suitable technique, for example, using any suitable eukaryotic or non-eukaryotic expression system. In certain embodiments, the antibody, antibody-based binding protein or antigen-binding fragment is produced using a mammalian expression system. Some specific techniques for generating the antibodies, antibody-based binding proteins or antigen-binding fragments or antigen-binding fragments of the invention are exemplified herein. In some embodiments, the antibodies, antibody-based binding proteins or antigen-binding fragments of the invention can be produced using a suitable non-eukaryotic expression system such as a bacterial expression system. Bacterial expression systems can be used to produce fragments such as a F(ab)2, Fv, scFv, IgGACH2, F(ab′)2, scFv2CH3, Fab, VL, VH, scFv4, scFv3, scFv2, dsFv, Fv, scFv-Fc, (scFv)2, and diabodies. Techniques for altering DNA coding sequences to produce such fragments are known in the art.

According to one preferred embodiment, the first and/or the second antibody, alternative scaffold or antibody mimetic is monospecific or mono epitope-reactive.

According to one embodiment of the respective aspect of the invention, the second antibody, alternative scaffold or antibody mimetic, or the second antigen-binding domain binds to ROR1, yet
to a different epitope than, and/or
does not compete for binding with

the antibody comprising a heavy chain variable region sequence shown in SEQ ID NO. 2 and a light chain variable region sequence shown in SEQ ID NO. 3.

According to one embodiment of the respective aspect of the invention, the second antibody, alternative scaffold or antibody mimetic, or antigen-binding domain
binds to the same ROR1 epitope as and/or
competes for ROR1 binding with

at least one antibody selected from the group consisting of
a) R12, comprising a heavy chain variable region sequence shown in SEQ ID NO. 20 and a light chain variable region sequence shown in SEQ ID NO. 21,
b) ERR1-TOP43, comprising a heavy chain variable region sequence shown in SEQ ID NO. 12 and a light chain variable region sequence shown in SEQ ID NO. 13,
c) ERR1-301, comprising a heavy chain variable region sequence shown in SEQ ID NO. 4 and a light chain variable region sequence shown in SEQ ID NO. 5,
d) ERR1-402, comprising a heavy chain variable region sequence shown in SEQ ID NO. 8 and a light chain variable region sequence shown in SEQ ID NO. 9, and/or
e) 2A2, comprising a heavy chain variable region sequence shown in SEQ ID NO. 22 and a light chain variable region sequence shown in SEQ ID NO. 23.

According to one embodiment of the invention, ROR1 as mentioned herein is human ROR1 (hROR1).

According to one embodiment of the respective aspect of the invention, the multispecific product is in a format selected from the group consisting of
bispecific or bi-epitope reactive scFv-Fc,
bispecific or bi-epitope reactive scFv-IgG, and/or
DVD-Ig.

The bispecific scFv-Fc format consists of two scFv fragments of different specificity genetically fused to an Fc fragment. The bispecific scFv-IgG format consists of an IgG shaped antibody with a given specificity with two scFv fragments of different specificity fused to the N-terminus of the VH domain of the IgG. The DVD-Ig format consists of an Ig shaped antibody with a given specificity, wherein each VL/VH pair carries, N-terminally, another VH/VL pair of different specificity.

Examples for the three formats are shown in the following table, and in FIG. 15

| | | | | |
|---|---|---|---|---|
| bispecific | V_{L}1 | V_{H}1 | V_{H}2 | V_{L}2 |
| scFv-Fc | | C_{H}2 | C_{H}2 | |
| | | C_{H}3 | C_{H}3 | |
| DVD-Ig | V_{L}2 | V_{H}2 | V_{H}2 | V_{L}2 |
| | V_{L}1 | V_{H}1 | V_{H}1 | V_{L}1 |
| | C_{L} | C_{H}1 | C_{H}1 | CL |
| | | C_{H}2 | C_{H}2 | |
| | | C_{H}3 | C_{H}3 | |
| bispecific | V_{L}2 | V_{H}2 | V_{H}2 | V_{L}2 |
| scFv IgG | V_{L}1 | V_{H}1 | V_{H}1 | V_{L}1 |
| | C_{L} | C_{H}1 | C_{H}1 | CL |
| | | C_{H}2 | C_{H}2 | |
| | | C_{H}3 | C_{H}3 | |

According to another embodiment, the first entity comprises the following CDRs:

(V_{L} CDR1, SEQ ID NO. 69)
QASQSVYGNNELA,
(V_{L} CDR2, SEQ ID NO. 70)
RASILTS,
(V_{L} CDR3, SEQ ID NO. 71)
LGGYVSQSYRAA,
(V_{H} CDR1 SEQ ID NO. 72)
RNGMT,
(V_{H} CDR2, SEQ ID NO. 73)
IITSSGDKYYATWAKG,
and
(V_{H} CDR3, SEQ ID NO. 74)
GTVSSDI,

These are the CDRs of antibody ERR1-324. The CDRs are comprised in a suitable protein framework so as to be capable to bind to ROR1.

According to another embodiment, the second entity comprises one of the following CDR sets:

| Antibody | 2A2 | XBR1-402 | R12 | TOP43 |
|---|---|---|---|---|
| VH CDR1 | GYTFSDYEMH | SYYMS | AYYMS | SYWMS |
| | (SEQ ID NO. 75) | (SEQ ID NO. 81) | (SEQ ID NO. 87) | (SEQ ID NO. 93) |
| VH CDR2 | AIDPETGGTAY | AIGISGNAYYASW | TIYPSSGKTYYATW | AIYGSGNTYYASW |
| | NQKFKG | AKS | VNG | AKG |
| | (SEQ ID NO. 76) | (SEQ ID NO. 82) | (SEQ ID NO. 88) | (SEQ ID NO. 94) |
| VH CDR3 | YYDYDSFTY | DIIPTYGMDL | DSYADDGALFNI | DVHSTATDL (SEQ |
| | (SEQ ID NO. 77) | (SEQ ID NO. 83) | (SEQ ID NO. 89) | ID NO. 95) |
| VL CDR1 | KASQNVDAAVA | EGNNIGSKAVH | TLSSAHKTDTID | GGNNIGSKAVN |
| | (SEQ ID NO. 78) | (SEQ ID NO. 84) | (SEQ ID NO. 90) | (SEQ ID NO. 96) |
| VL CDR2 | SASNRYT | DDDERPS | GSYTKRP | NDDERPS |
| | (SEQ ID NO. 79) | (SEQ ID NO. 85) | (SEQ ID NO. 91) | (SEQ ID NO. 94) |
| VL CDR3 | QQYDIYPYT | QVWDSSAYV | GADYIGGYV | QLWDSSAGAYV |
| | (SEQ ID NO. 80) | (SEQ ID NO. 86) | (SEQ ID NO. 92) | (SEQ ID NO. 98) |

Again, the CDRs are comprised in a suitable protein framework so as to be capable to bind to ROR1.

According to another embodiment, the first entity comprises the heavy chain variable region sequence of antibody ERR1-324 shown in SEQ ID NO. 2 and the light chain variable region sequence of antibody ERR1-324 shown in SEQ ID NO. 3.

According to another embodiment, the second entity comprises at least one of the following sequence pairs:
a) the heavy chain variable region sequence of antibody R12 shown in SEQ ID NO. 20 and the light chain variable region sequence shown in SEQ ID NO. 21,
b) the heavy chain variable region sequence of antibody ERR1-TOP43 shown in SEQ ID NO. 12 and the light chain variable region sequence of antibody ERR1-TOP43 shown in SEQ ID NO. 13,
c) the heavy chain variable region sequence of antibody ERR1-301 shown in SEQ ID NO. 4 and the light chain variable region sequence of antibody ERR1-301 shown in SEQ ID NO. 5,
d) the heavy chain variable region sequence of antibody ERR1-402 shown in SEQ ID NO. 8 and the light chain variable region sequence of antibody ERR1-402 shown in SEQ ID NO. 9, and/or
e) the heavy chain variable region sequence of antibody 2A2 shown in SEQ ID NO. 22 and the light chain variable region sequence of antibody 2A2 shown in SEQ ID NO. 23.

According to another aspect of the invention, an antibody drug conjugate (ADC) having the general formula A-(L)n-(T)m is provided, in which
A is at least one antigen binding domain, antibody, alternative scaffold or antibody mimetic according to the above description,
L is a linker,
T is a toxin

and in which n and m are integers between >1 and <10.

According to another aspect of the invention, an antibody effector conjugate (AEC) having the general formula A-(L)n-(E)m is provided, in which
A is at least one antigen binding domain, antibody, alternative scaffold or antibody mimetic according to the above description,
L is a linker,
E is a label

and in which n and m are integers between >1 and <10.

Such label can be a detectable label, can be at least one selected from the group consisting of: a fluorescent label (including a fluorescent dye or a fluorescent protein), a chromophore label, a radioisotope label containing iodine (e.g., ¹²⁵I), gallium (⁶⁷Ga), indium (¹¹¹I), technetium (^{99m}Tc), phosphorus (³²P), carbon (¹⁴C), tritium (³H), other radioisotope (e.g., a radioactive ion), and/or a protein label such as avidin or streptavidin.

According to one embodiment of the respective aspect of the invention, the antibody is a multi-specific, preferably a bi-epitope reactive antibody according to the above description.

According to another aspect of the invention, a composition comprising at least two antibody effector conjugates (AEC) or antibody drug conjugates (ADC) according to the above description, wherein each of the two conjugates comprises one of the monospecific antibodies, alternative scaffolds or antibody mimetics according to the above description.

According to yet another embodiment of the invention, the ADC is a bi-epitope reactive ADC (abbreviated BETR-ADC™) binding to two different epitopes of the ROR1 target.

According to one embodiment of the respective aspect of the invention, the linker is at least one selected from the group consisting of
an oligopeptide linker
a maleimide linker, optionally comprising cleavable spacers, that may be cleaved by changes in pH, redox potential and or specific intracellular or extracellular enzymes.

According to one embodiment, the linker has at least one of the following amino acid sequences: -LPXTGn-, -LPXAGn-, -LPXSGn-, -LAXTGn-, -LPXTGn-, -LPXTAn- or -NPQTGn-, with Gn being an oligo- or polyglycine with n being an integer between ≥1 and ≤21, An being an oligo- or polyalanine with n being an integer between ≥1 and ≤21, and X being any conceivable amino acid sequence.

Gn (also called Gly(n)) is the oligoglycin discussed elsewhere herein In a preferred embodiment its length n can be between ≥1 and ≤21, preferably between ≥1 and ≤5.

It is important to understand that, in one specific embodiment (where Streptococcus pyogenes sortase A is used, see below), the oligo-glycine (Gly)ₙ can optionally be replaced by an oligo-alanine (Ala)ₙ.

According to one embodiment of the respective aspect of the invention, the linker is conjugated to the C-terminus of at least one subdomain of the antibody.

According to one embodiment of the respective aspect of the invention, prior to conjugation
the antibody bears a sortase recognition tag used or conjugated to the C-terminus of at least one subdomain thereof, and
the toxin or label comprises a glycine stretch with a length of between ≥1 and ≤20 glycine residues, preferably with a length of ≥2 and ≤5 glycine residues.

According to another embodiment, said sortase enzyme recognition motif comprises at least one of the following amino acid sequences: LPXTG, LPXAG, LPXSG, LAXTG, LPXTA or NPQTN, with X being any conceivable amino acid sequence.

The following table shows the recognition tags and the peptides derived therefrom to be part of the linker:

| | |
|---|---|
| Staphylococcus aureus sortase A recognition sequence, | -LPXTG |
| with X being any amino acid | |
| Staphylococcus aureus sortase A recognition sequence, | -LPXAG |
| with X being any amino acid | |
| recognition sequence for Staphylococcus aureus sortase | -LPXSG |
| A or engineered sortase A 4S-9 from Staphylococcus | |
| aureus, with X being any amino acid | |
| recognition sequence for engineered sortase A 2A-9 from | -LAXTG |
| Staphylococcus aureus, with X being any amino acid | |
| Streptococcus pyogenes sortase A recognition sequence, | -LPXTA |
| with X being any amino acid | |
| Staphylococcus aureus sortase recognition sequence | -NPQTN |
| Linker derived from Staphylococcus aureus sortase A | -LPXT(Gn)- |
| recognition sequence, with X being any amino acid and | |
| n ≥ 1 and ≤21 | |
| Linker derived from Staphylococcus aureus sortase A | -LPXA(Gn)- |
| recognition sequence, with X being any amino acid and | |
| n ≥ 1 and ≤21 | |
| Linker derived from recognition sequence for | -LPXS(Gn)- |
| Staphylococcus aureus sortase A or engineered sortase | |
| A 4S-9 from Staphylococcus aureus, with X being any | |
| amino acid and n ≥ 1 and ≤21 | |
| Linker derived from recognition sequence for engineered | -LAXT(Gn)- |
| sortase A 2A-9 from Staphylococcus aureus, with X being | |
| any amino acid | |
| and n ≥1 and ≤21 | |
| Linker derived from Streptococcus pyogenes sortase A | -LPXT(Gn)- |
| recognition sequence, with X being any amino acid and | or |
| n ≥ 1 and ≤21 | -LPXT(An)- |
| Linker derived from Staphylococcus aureus sortase | -NPQT(Gn)- |
| recognition sequence, with n ≥ 1 and ≤21 | |

Engineered sortases, including but not limited to sortase A mutant 2A-9 and sortase A mutant 4S-9 from Staphylococcus aureus, are described in Dorr et al. (2014) and mutants described in Chen et al. (2011).

As background and to exemplify the general concept of sortase transpeptidation, Sortase A uses an oligo-glycine-stretch as a nucleophile to catalyze a transpeptidation by which the terminal amino group of the oligo-glycine effects a nucleophilic attack on the peptide bond joining the last two C-terminal residues of the sortase tag. This results in breakage of that peptide bond and formation of a new peptide bond between the C-terminally second-to-last residue of the sortase tag and the N-terminal glycine of the oligo-glycine peptide, i.e. resulting in a transpeptidation.

It is important to understand that, in one specific embodiment (where Streptococcus pyogenes sortase A is used, see above), the oligo-glycine (Gly)n can optionally be replaced by an oligo-alanine (Ala)n.

Prior to sortase conjugation, the sortase recognition motif may, at its C-terminus, furthermore carry other tags, like His-tags, Myc-tags or Strep-tags (see FIG. 4a of WO 2014/140317, the content of which is incorporated by reference herein). However, because the peptide bond between the 4th and 5th amino acid of the sortase tag is cleaved upon sortase A mediated conjugation, these additional tags do not appear in the conjugated product.

The sortase tag may, for example, be fused to a C-terminus of a binding protein, or to a domain or subunit thereof, by genetic fusion and co-expressed therewith. In another preferred embodiment, the sortase tag may directly be appended to the last naturally occurring C-terminal amino acid of the immunoglobulin light chains or heavy chains, which in case of the human immunoglobulin kappa light chain is the C-terminal cysteine residue, and which in the case of the human immunoglobulin IgG1 heavy chain may be the C-terminal lysine residue encoded by human Fcγ1 cDNA. However, another preferred embodiment is also to directly append the sortase tag to the second last C-terminal glycine residue encoded by human Fcγ1 cDNA, because usually terminal lysine residues of antibody heavy chains are clipped off by posttranslational modification in mammalian cells. Therefore, in more than 90% of the cases naturally occurring human IgG1 lacks the C-terminal lysine residues of the IgG1 heavy chains.

Therefore, one preferred embodiment of the invention is to omit the C-terminal lysine amino acid residues of human IgG1 heavy chain constant regions in expression constructs for sortase recognition-motif tagged Igγ1 heavy chains. Another preferred embodiment is to include the C-terminal lysine amino acid residues of human IgG1 heavy chain constant regions in expression constructs for sortase recognition-motif tagged Igγ1 heavy chains.

In another preferred embodiment the sortase or oligoglycine tag may be appended to the C-terminus of a human immunoglobulin IgG1 heavy chain where the C-terminal lysine residue encoded by human Fcγ1 cDNA is replaced by an amino acid residue other than lysine to prevent unproductive reactions of sortase with the ε-amino group of said C-terminal lysine residue leading to inter-heavy chain crosslinking.

We have described previously that in some cases (e.g. at the C-terminus of the Ig kappa light chains, see: Beerli et al. (2015) PloS One 10, e131177) it is beneficial to add additional amino acids between the C-terminus of the binding protein and the sortase tag. This has been shown to improve sortase enzyme conjugation efficiencies of payloads to the binding protein. In the case of Ig kappa light chains, it was observed that by adding 5 amino acids between the last C-terminal cysteine amino acid of the Ig kappa light chain and the sortase pentapeptide motif improved the kinetic of conjugation, so that the C-termini of Ig kappa light chains and Ig heavy chains could be conjugated with similar kinetics (see: Beerli et al. (2015) PloS One 10, e131177). Therefore, it is another preferred embodiment that optionally ≥1 and ≤11 amino acids are added in between the last C-terminal amino acid of a binding protein or antibody subunit and the sortase tag. In a preferred embodiment, a GₙS peptide (wherein n is from 1 to 10, preferably 1 to 5) is added between the last C-terminal amino acid of a binding protein or antibody subunit and the sortase tag. Finally, in another preferred embodiment, additional amino acids between the C-terminus of the binding protein and the sortase or oligoglycine tag may beneficially be included that comprise a sequence and/or linker that is cleavable by hydrolysis, by a pH change or by a change in redox potential, or that is cleavable by a non-sortase enzyme, e.g., by proteases.

According to one embodiment of the respective aspect of the invention, the toxin or derivative thereof is at least one selected from the group consisting of:
maytansinoids,
auristatins,
anthracyclins, preferably PNU-derived anthracyclins
calicheamicins,
tubulysins
duocarmycins
radioisotopes
liposomes comprising a toxid payload
protein toxins
taxanes, and/or
pyrrolobenzodiazepines.

In a preferred embodiment of the ADC, the toxin is selected from PNU-159682 as described in Quintieri et al. (2005) and derivatives thereof, maytansine, monomethyl auristatin MMAE, and monomethyl auristatin MMAF. In a preferred embodiment of the ADC, the toxin, joined to the linker at its wavy line, is of formula (i), as described in WO 2016/102679:

[chemical structure]

In the embodiment where the toxin is of formula (i), the linker may optionally comprise an alkyldiamino group of the form NH₂—(CH₂)ₘ—NH₂, where m≥1 and ≤11, preferably m=2, such that one amino group is directly linked at the wavy line of formula (i) to form an amide bond. It is moreover preferred that the second amino group is linked to an oligopeptide linker, which is more preferably an oligoglycine.

According to one embodiment of the respective aspect of the invention, the conjugate is created by sortase-mediated conjugation of (i) an antibody carrying one or more sortase recognition tags and (ii) one or more toxins or labels carrying an oligoglycine tag.

According to another aspect of the invention, a method of producing a conjugate according to the above description is provided, which method comprises the following steps:

a) providing an antibody, alternative scaffold or antibody mimetic according to the above description, which antibody carries a sortase recognition tag,

b) providing one or more toxins or labels carrying an oligoglycine tag, and

c) conjugating the antibody, alternative scaffold or antibody mimetic and the toxin or label by means of sortase-mediated

conjugation.

According to another embodiment of the invention, the use of the multispecific product according to the above description or the antibody drug conjugate according to the above description, for the treatment of a patient that is
suffering from,
at risk of developing, and/or
being diagnosed for

a neoplastic disease is provided.

As an alternative, a method of treating a patient suffering from, at risk of developing, and/or being diagnosed for a neoplastic disease is provided, which method comprises the administration of one or more therapeutically active doses of the multispecific product according to the above description or the antibody drug conjugate according to the above description.

According to another aspect of the invention, the neoplastic disease is a neoplastic disease characterized by expression of ROR1. In one embodiment, ROR1 is overexpressed in said neoplastic disease.

As used herein, the term “overexpression of ROR1” refers to the expression level of ROR1 mRNA and/or protein expressed in cells of a given tissue being elevated in comparison to the levels of ROR1 as measured in normal cells (free from disease) of the same type of tissue, under analogous conditions. Said ROR1 mRNA and/or protein expression level may be determined by a number of techniques known in the art including, but not limited to, quantitative RT-PCR, western blotting, immunohistochemistry, and suitable derivatives of the above.

According to another aspect of the invention, the neoplastic disease is at least one selected from the group consisting of sarcoma, renal cell carcinoma, breast cancer, incl. triple-negative breast cancer, lung cancer, colon carcinoma, testicular cancer, ovarian cancer, pancreatic cancer, kidney cancer, gastric cancer, prostate cancer, head and neck cancer, melanoma, squamous cell carcinoma, multiple myeloma and other cancers, mesothelioma, chronic lymphoblastic leukemia, mantle cell lymphoma, non-Hodgkin lymphoma, Hodgkin lymphoma, preB acute lymphocytic leukemia, acute myeloid leukemia, multiple myeloma, and other types of leukemias and lymphomas as well as solid tumors.

According to another aspect of the invention, a pharmaceutical composition comprising the multi-specific product according to the above description, the antibody drug conjugate according to the above description together with one or more pharmaceutically acceptable ingredients, is provided.

According to another aspect of the invention, a method of killing or inhibiting the growth of a cell expressing or overexpressing ROR1 in vitro or in a patient is provided, which method comprises administering to the cell a pharmaceutically effective amount or dose of the multi-specific or bi-epitope reactive product according to the above description, the antibody drug conjugate according to the above description, or of the pharmaceutical composition according to the above description.

According to one embodiment, the cell expressing ROR1 is a cancer cell.

### EXAMPLES

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

All amino acid sequences disclosed herein are shown from N-terminus to C-terminus; all nucleic acid sequences disclosed herein are shown 5′->3′.

### Example 1. Establishment of 63-12 Cells Stably Expressing hROR1 or hROR2

The mouse Abelson murine pre-B cell line 63-12 (Shinkai et al. (1992) Cell 68:855-67) was cultured in culture media (17.7 g/L Gibco® IMDM (Life Technologies, 42200-030), 3.024 g/L NaHCO₃(Sigma-Aldrich, p.a., >99.7%), 10 mL/L 100× non-essential amino acids (Life Technologies, 11140035), 5 mg/L insulin (Sigma-Aldrich, 1-5500), 3 mL/L of 10% primatone RL/UF in H₂O (Sheffield Bioscience), and 1 mL/L of 50 mM 2-mercaptoethanol (Sigma-Aldrich, M-3148) in H₂O), supplemented with 2% (v/v) FCS, 100 IU/mL Pen/Strep/Fungizone (Amimed, 4-02F00-H), 200 mM L-glutamine (Amimed, 5-10K00-H) and 50 μM 2-mercaptoethanol (Amresco, 0482) at 37° C. and 7.5% CO2.

Cells were engineered to overexpress hROR1 and hROR2 by transposition as follows: cells were centrifuged (6 min, 1200 rpm, 4° C.) and resuspended in RPMI-1640 media (5×10⁶ cells/mL). 400 μL of cell suspension was then added to 400 μL of RPMI containing 10 μg of transposable vector pPB-PGK-Puro-ROR1 (directing co-expression of full-length ROR1 (NP 005003.2) and the puromycin-resistance gene), or 10 μg of transposable vector pPB-PGK-Puro-ROR2 (directing co-expression of full-length ROR2 (NP_004551.2) and the puromycin-resistance gene), along with 10 μg of transposase-containing vector pCDNA3.1_hy_mPB. DNA/63-12 cell mixtures were transferred to electroporation cuvettes (0.4 cm-gap, 165-2088, BioRad, Cressier, Switzerland) and electroporated using the Biorad Gene Pulser II with capacitance extender at 300V and 950 μf. Then, cells were incubated for 5-10 min at room temperature. Following the incubation, cells were centrifuged at 1200 rpm for 6 min (4° C.), washed once and subsequently resuspended in aqueous culture media (17.7 g/L Gibco® IMDM (Life Technologies, 42200-030), 3.024 g/L NaHCO₃(Sigma-Aldrich, p.a., >99.7%), 10 mL/L 100× non-essential amino acids (Life Technologies, 11140035), 5 mg/L insulin (Sigma-Aldrich, 1-5500), 3 mL/L of 10% primatone RL/UF in H₂O (Sheffield Bioscience), and 1 mL/L of 50 mM 2-mercaptoethanol (Sigma-Aldrich, M-3148) in H₂O), supplemented with 2% (v/v) FCS, 100 IU/mL Pen/Strep/Fungizone (Amimed, 4-02F00-H), 200 mM L-glutamine (Amimed, 5-10K00-H) and 50 μM 2-mercaptoethanol (Amresco, 0482). After two days incubation at 37° C. in a humidified incubator at 5% CO2 atmosphere, cell pools stably expressing hROR1 or hROR2 were selected by adding 2 μg/mL puromycin (Sigma-Aldrich, P8833).

After 4 to 5 days, hROR1 or hROR2 expression on engineered cells were confirmed by flow cytometry. Briefly, following trypzinization, 10⁶ cells were centrifuged in FACS tubes; obtained pellets were resuspended in buffer (PBS with 2% (v/v) FCS). In the case of hROR1-engineered cells, cells were then incubated with 2A2 (mAb066 antibody targeting ROR1, final concentration 2 μg/mL) for 30 min at 4° C., followed by centrifugation and washing. Cells were resuspended as previously and incubated with anti-human IgG antibody (Fc gamma-specific) PE (eBioscience, Vienna, Austria, 12-4998-82), at a 1:100 dilution, in the dark (30 min, 4° C.), washed once in buffer and kept on ice until FACS sorting. For hROR2-engineered 63-12 cells, the same protocol was followed but using EPR3779 (Abcam antibody targeting ROR2; 1:100 dilution) as primary antibody and allophycocyanin-conjugated AffiniPure F(ab′)2 goat anti-rabbit IgG (H+L) (Jackson Immunoresearch, 111-136-144) as secondary antibody.

In the case of hROR1-engineered 63-12 cells, cells were single cell sorted into 96-well flat-bottom plates containing 200 μL of supplemented culture media per well using a FACS Aria II. Plates were incubated at 37° C. and clones were expanded to 6-well plates before analysis. Target expression was confirmed by flow cytometry using a FACSCalibur instrument (BD Biosciences) and FlowJo analytical software (Tree Star, Ashland, Oreg.).

63-12, 63-12/hROR1 and 63-12/hROR2 transfectants were cultured in DMEM (Invitrogen; Carlsbad, Calif.) supplemented with 10% (v/v) heat inactivated FBS (Thermo Scientific; Logan, Utah), 100 IU/mL penicillin, and 100 mg/mL streptomycin (Invitrogen). HEK 293F cells were purchased from Invitrogen and maintained in FreeStyle Medium supplemented with 1% (v/v) heat inactivated FBS (Thermo Scientific) to support adherent culture or without FBS for suspension culture, 100 U/mL penicillin, and 100 mg/mL streptomycin (Invitrogen).

### Example 2. Generation of High-Complexity Rabbit Fab Library and Reagents for Screening

Construction, expression, and purification of recombinant human ROR1 proteins: Construction, expression, purification and biotinylation of hFc fusion proteins containing different domains of human ROR1 or mouse ROR1 were described (Yang et al., PloS One 6:e21018, 2011). For hROR1-AVI-6×HIS fusion protein, the extracellular domain of human ROR1 (24-403) was PCR amplified with primers pCEP4-hROR1-F and pCEP4-hROR1-Avi tag-R (note that the AVI tag was introduced to the C terminus of ROR1 by primer pCEP4-hROR1-Avi tag-R), followed by extension PCR with primers pCEP4-signal-F-KpnI and pCEP4-6HIS-R-XhoI to add a signal peptide and 6×HIS tag to the N and C terminus separately before cloning into pCEP4 via KpnI/XhoI. This construct was then transiently transfected into HEK 293F cells (Invitrogen) using 293fectin (Invitrogen), and the protein was purified by Immobilized Metal Ion Affinity Chromatography using a 1-mL HisTrap column (GE Healthcare) as described in Kwong and Rader, Curr Protoc Protein Sci Chapter 6:Unit 6 10, 2009. The quality and quantity of purified hROR1-AVI-6×HIS was analyzed by SDS-PAGE and A₂₈₀ absorbance, respectively. Subsequently, the fusion protein was biotinylated by BirA enzyme kit from Avidity (Aurora, Colo.) following the protocol. Briefly, 2 mg ROR1-AVI-6×HIS at 40 μM in 10 mM Tris-HCl (pH 8) was biotinylated in the presence of biotin using 10 μg BirA after incubation for 30 min at 37° C., followed by purification again using a 1-mL HisTrap column (GE Healthcare) as described above.

pCEP4-hROR1-F:
(SEQ ID NO: 26)
5′-atcctgtttctcgtagctgctgcaactggagcacactccgcccggg
gcgccgccgcccag-3′;
pCEP4-hROR1-Avi-tag-R:
(SEQ ID NO: 27)
5′-ccactcgatcttctgggcctcgaagatgtcgttcaggccctccatc
ttgttcttctcctt-3′;
pCEP4-signal-F-KpnI:
(SEQ ID NO: 28)
5′ gctgggtaccggcgcgccaccatggactggacttggagaatcctgt
ttctcgtagctgct-3′;
pCEP4-6HIS-R-XhoI:
(SEQ ID NO: 29)
5′-gccggcctcgagtcagtgatggtgatggtggtgctcgtgccactcg
atcttctgggcctc-3′.

Construction, expression, and purification of recombinant human ROR1 (hROR1-His) and human ROR2 (hROR2-His) proteins: hROR1-His was PCR-amplified with primers

SP-hROR1_F
(SEQ ID NO: 50)
(5′ gctgggtaccggcgcgccaccatggactggacttggagaatcctg
tttctcgtagctgctgcaactggagcacactccgcccggggcgccgccg
cccag 3′)
and
hROR1-His_R
(SEQ ID NO: 30)
(5′ cggcctcgagtcagtgatggtgatggtggtgctccatcttgttct
tctcctt 3′)

using pCEP4-hFc-hROR1 (Yang et al., PloS One 6:e21018, 2011) as template, while hROR2-His was PCR-amplified with primers

SP-hROR2_F
(SEQ ID NO: 31)
(gctgggtaccggcgcgccaccatggactggacttggagaatcctgttt
ctcgtagctgctgcaactggagcacactccgaagtggaggttctggatc
cg)
and
hROR2-His_R
(SEQ ID NO: 32)
(cggcctcgagtcagtgatggtgatggtggtgccccatcttgctgctgt
ctcg)

using pCEP4-hFc-hROR2 as template. Then they are cloned into pCEP4 (Invitrogen) separately via KpnI/XhoI. These constructs were then separately and transiently transfected into HEK 293F cells (Invitrogen) using 293fectin (Invitrogen), and the corresponding proteins were purified by Immobilized Metal Ion Affinity Chromatography using a 1-mL HisTrap column (GE Healthcare) as described in Kwong and Rader, Curr Protoc Protein Sci Chapter 6:Unit 6 10, 2009. The quality and quantity of purified hROR1-His and hROR2-His were analyzed by SDS-PAGE and A₂₈₀ absorbance, respectively.

Generation and selection of naïve chimeric rabbit/human Fab libraries: All rabbit handling was carried out by veterinary personnel at Pocono Rabbit Farm & Laboratory (Canadensis, Pa.) or R & R Research (Stanwood, Wash.). A total of nine rabbits (ages 3-4 months) were used. Five of these rabbits were of the New Zealand White (NZW) strain, with three obtained from Pocono Rabbit Farm & Laboratory (Canadensis, Pa.) and two obtained from R & R Research (Stanwood, Wash.). Four b9 wild-type rabbits were derived from a separate R & R Research colony that originated from a pedigreed colony developed and characterized at the National Institute of Allergy and Infectious Diseases (NIAID) (McCartney-Francis et al., Proc. Natl. Acad. Sci. USA 81:1794-1798, 1984; and Popkov et al., J. Mol. Biol. 325:325-335, 2003. Spleen and bone marrow from each rabbit were collected and processed for total RNA preparation and RT-PCR amplification of rabbit V_{κ}, V_{λ}, and V_{H} encoding sequences using established protocols (Rader, Methods Mol Biol 525:101-128, xiv, 2009. Rabbit (rb) V_{κ}/human (hu) C_{κ}/rbV_{H} and rbV_{λ}/huC_{λ}/rbV_{H} segments, respectively, were assembled in one fusion step based on 3-fragment overlap extension PCR. Note that the V_{L} derived from b9 rabbits were also assembled with V_{H} from NZW rabbits. The Fab-encoding fragments were digested with SfiI and ligated with SfiI-treated phage display vector pC3C (Hofer et al., J Immunol Meth 318:75-87, 2007) at 16° C. for 24 h. Subsequently, 15 μg purified pC3C-rbV_{κ}/hC_{κ}/rbV_{H} ligated products were transformed into E. coli strain SR320 (a kind gift from Dr. Sachdev S. Sidhu, University of Toronto, Toronto, Ontario, Canada) by 30 separate electroporations (each using 0.5 μg DNA in 50 μl electrocompetent cells) and yielded 7.5×10⁹ independent transformants for library κ. For library λ, 4.8×10⁹ independent transformants were obtained using the same procedure. Using VCSM13 helper phage (Stratagene; La Jolla, Calif.), the phagemid libraries were converted to phage libraries and stored at −80° C. Phage library κ and library λ were re-amplified using XL1-Blue (Stratagene) or ER2738 (Lucigen) and mixed equally before four rounds of panning against biotinylated hFc-hROR1 or hROR1-AVI-6HIS. During the panning, 5 μg/mL antigen was pre-incubated with streptavidin coated magnetic beads (Dynabeads MyOne Streptavidin Cl; Invitrogen) at 37° C. for 30 min and then binders from the phage library were captured in the presence of 1 mg/mL unspecific polyclonal human IgG (Thermo Scientific) when hFc-ROR1 was used. Starting from the third round of panning, the input phage was negatively depleted by incubation with empty beads before selection against antigen-loaded beads. Following selection, supernatants of IPTG-induced bacterial clones were analyzed by ELISA and by flow cytometry. Repeated clones were identified by DNA fingerprinting with AluI, and the V_{L} and V_{H} sequences of unique clones were determined by DNA sequencing (FIG. 1).

### Example 3. Expression and Purification of Chimeric Rabbit/Human Fab and Full-Length IgG1 Antibodies

Construction, expression, and purification of chimeric rabbit/human Fab and IgG1: MAb XBR1-402 in chimeric rabbit/human Fab format was cloned into E. coli expression plasmid pC3C-His and expressed and purified as described in Kwong and Rader, Curr Protoc Protein Sci Chapter 6:Unit 6 10, 2009. For the expression of mAb XBR1-402 in chimeric rabbit/human IgG1 format, the previously described vector PIGG-R11 was used (Yang et al., PloS One 6:e21018, 2011). The V_{H} encoding sequence of Fab XBR1-402 was PCR amplified using primers XBR1-402_VH_F and XBR1-402_VH_R, and cloned via ApaI/SacI into PIGG-R11. Then the light chain encoding sequence of XBR1-402 was PCR amplified using primers XBR1-402_λ_F and LEAD-B, and cloned via HindIII/XbaI into PIGG-R11 with the corresponding heavy chain encoding sequence. Note that an internal ApaI site in FR4 of V_{H} encoding sequences of Fab XBR1-402 was removed by silent mutation in primer XBR1-402_VH_R. In addition, we changed a TAG stop codon, which was suppressed during selection in E. coli strain XL1-Blue, to CAG (glutamine) encoding the first amino acid of native V_{H} (FIG. 1) with primer XBR1-402_VH_F. The resulting PIGG-XBR1-402 plasmid was transiently transfected into HEK 293F cells (Invitrogen) using 293fectin (Invitrogen), and the protein purified with a 1-mL recombinant Protein A HiTrap column (GE Healthcare, Piscataway, N.J.) as described (Yang et al., PloS One 6:e21018, 2011; and Yang and Rader, Methods Mol Biol 901:209-232, 2012). The quality and quantity of purified IgG1 were analyzed by SDS-PAGE and A₂₈₀ absorbance, respectively.

All the other mAbs in chimeric rabbit/human Fab format were cloned into E. coli expression plasmid pET11a and expressed and purified as described (Yang et al., PloS One 6:e21018, 2011). For the expression of mAbs ERR1-324, ERR1-TOP43 and ERR1-TOP54 in chimeric rabbit/human IgG1 format, pCEP4 (Invitrogen) was used to clone the heavy chain and light chain separately. For heavy chain, a gBlock containing a heavy-chain signal peptide encoding sequence, V_{H} of ERR2-302 (a mAb to hROR2) and CH1 (1-49) of human IgG1 was synthesized by IDT (San Jose, Calif.) and amplified with primers KpnI/AscI-Signal and CH1-internal/overlap-R, and fused to CH1 (50-88)-CH2-CH3 amplified from PIGG with primers CH1-internal/overlap-F and HC-CH3-R-XhoI by overlap extension PCR with primers KpnI/AscI-Signal and HC-CH3-R-XhoI, and then cloned into pCEP4 by AscI/XhoI. Note that a EheI site was introduced into CH1 at Ala¹² by synonymous mutation when the gBlock was synthesized. Consequently, this construct served as vector to clone the heavy chains of other mAbs by replacing the V_{H} using AscI/EheI: V_{H} of ERR1-324, ERR1-TOP43 and ERR1-TOP54 were amplified with forward primer ERR1-324 HC-F, ERR1-TOP43 HC-F and ERR1-TOP54 HC-F and reverse primer VH-CH1-R-EheI separately, followed by extension PCR to add the signal peptide with primer KpnI/AscI-Signal and VH-CH1-R-EheI. Then, each V_{H} was inserted into the vector by AscI/EheI. For light chain cloning, while lambda light chains of ERR1-TOP43 and ERR1-TOP54 were amplified with primers ERR1-TOP43 LC-F and ERR1-TOP54 LC-F separately combined with LC-R-XhoI, kappa light chains of ERR1-324 was amplified with primers ERR1-324 KC-F and KC-R-XhoI. Then, a signal peptide encoding sequence was added by extension PCR with forward primer KpnI/AscI-Signal and reverse primer LC-R-XhoI or KC-R-XhoI. Subsequently, each light chain PCR product was cloned into pCEP4 by AscI/XhoI. The resulting constructs containing heavy chain or light chain for each IgG were co-transfected transiently into HEK293F cells (Invitrogen) using 293fectin (Invitrogen), and the corresponding proteins were purified with a 1-mL recombinant Protein A HiTrap column (GE Healthcare, Piscataway, N.J.) as described (Yang et al., PloS One 6:e21018, 2011; and Yang and Rader, Methods Mol Biol 901:209-232, 2012). The quality and quantity of purified IgG1 was analyzed by SDS-PAGE and A₂₈₀ absorbance, respectively.

TABLE 1
Primer sequences for cloning antibody sequences

| | | SEQ |
|---|---|---|
| | | ID |
| Primer | Sequence | NO: |
| XBR1-402_VH_F | GAGGAGGAGCTCACTCTCAGGAGCAGCAGAAG | 33 |
| | GAGTCCGGG | |
| XBR1-402_VH_R | CGATGGGCCCTTGGTGGAGGCTGAAGAGACGG | 34 |
| | TGACGAGGGTCCCTGGCCCCCAGAGGTC | |
| XBR1-402_λ_F | GAGAAGCTTGTTGCTCTGGATCTCTGGTGCCT | 35 |
| | ACGGGTCCTATGAGCTGACACAGCTGCC | |
| LEAD-B | GGCCATGGCTGGTTGGGCAGC | 36 |
| KpnI/AscI- | GGTACCGGCGCGCCACCATGGACTGGACTTGG | 37 |
| Signal | AGAATCCTGTTTCTCGTAGCTGCTGCAA | |
| CH1-internal/ | GCCGCTGGTCAGGGCTCCTG | 38 |
| overlap-R | | |
| CH1-internal/ | CAGGAGCCCTGACCAGCGGC | 39 |
| overlap-F | | |
| HC-CH3-R-XhoI | GGCCTCGAGTCATTTACCCGGAGACAGGGA | 40 |
| ERR1-324 HC-F | TTTCTCGTAGCTGCTGCAACTGGAGCACACTC | 41 |
| | CCAGTCGCTGGAGGAGTCCGGG | |
| ERR1-TOP43 | TTTCTCGTAGCTGCTGCAACTGGAGCACACTC | 42 |
| HC-F | CCAGTCGTTGGAGGAGTCCGGG | |
| ERR1-TOP54 | Same as ERR1-TOP43 HC-F | 43 |
| HC-F | | |
| VH-CH1-R-EheI | GGAGGGCGCCAGGGGGAAGACCGATGGGCCCT | 44 |
| | TGGT | |
| ERR1-TOP43 | TTTCTCGTAGCTGCTGCAACTGGAGCACACTC | 45 |
| LC-F | CTCCTATGAGCTGACACAGCTG | |
| ERR1-TOP54 | Same as ERR1-TOP43 LC-F | 46 |
| LC-F | | |
| ERR1-324 KC-F | TTTCTCGTAGCTGCTGCAACTGGAGCACACTC | 47 |
| | CGAGCTCGTGCTGACCCAGACT | |
| LC-R-XhoI | GGCCTCGAGTTATGAACATTCTGTAGGGGC | 48 |
| KC-R-XhoI. | GGCCTCGAGTTAACACTCTCCCCTGTTGAA | 49 |

### Example 4. Examination of Antibody Binding Activities

ELISA: For ELISA (FIG. 2), each well of a 96-well Costar 3690 plate (Corning, Corning, N.Y.) was coated with 100 ng anti-human IgG1 Fc in 25 μL coating buffer (0.1M Na₂CO₃, 0.1M NaHCO₃, pH 9.6) for 1 h at 37° C. After blocking with 150 μL 3% (w/v) BSA/TBS for 1 h at 37° C., hFc-hROR1 or hFc-mROR1 was captured following incubation at 100 ng/50 μL for 1 h at 37° C. Then 100 ng/50 μL of Fab was applied in each well at 37° C. 2 h later, 50 μL of a 1:1000 dilution of a mouse anti-His tag mAb conjugated to horseradish peroxidase (HRP) (R&D Systems, Minneapolis, Minn.) in 1% (w/v) BSA/TBS was used to detect the Fab.

To determine the epitopes (FIG. 4), hFc fusion proteins containing different domains of hROR1 were coated directly, followed by incubation with Fab before detection by mouse anti-His tag mAb conjugated to HRP. Washing with PBS was repeated and colorimetric detection was performed using 2,2′-azino-bis (3-ethylbenzthiazoline)-6-sulfonic acid (Roche) as substrate according to the manufacturer's directions. The absorbance was measured at 405 nm using a SpectraMax M5 microplate reader (Molecular Devices; Sunnyvale, Calif.) and SoftMax Pro software (Molecular Devices).

Flow cytometry: Cells were stained using standard flow cytometry methodology. Briefly, for purified anti-ROR1 Fab (FIG. 3), 0.1-1×10⁶ cells were stained with 1000 ng/100 μL of Fab on ice for 1 h. After washing twice with ice-cold flow cytometry buffer (PBS containing 1% (v/v) BSA, 0.1% sodium azide and 1 mM EDTA), the cells were incubated with a 1:1000 dilution of a mouse anti-His tag mAb conjugated to Alexa Fluor 488 (Qiagen) in 100 μL flow cytometry buffer on ice for 30 min.

Surface plasmon resonance: Surface plasmon resonance for the measurement of the affinities of all Fabs to hFc-hROR1 and for epitope mapping studies were performed on a Biacore X100 instrument using Biacore reagents and software (GE Healthcare, Piscataway, N.J.). Anti-Human IgG (Fc) antibody was immobilized on a CMS sensor chip following the instruction of Human Antibody Capture Kit (GE Healthcare, Piscataway, N.J.). Then, hFc-hROR1 fusion proteins were captured at certain density (indicated in FIG. 5). The sensor chip included an empty flow cell for instantaneous background depletion. All binding assays used 1×HBS-EP+ running buffer (10 mM HEPES, 150 mM NaCl, 3 mM EDTA (pH 7.4), and 0.05% (v/v) Surfactant P20) and a flow rate of 30 mL/min. For affinity measurements, all Fabs were injected at five different concentrations (dilution factor was 2), and the lowest concentration was tested in duplicates (the highest concentrations for each Fab are indicated in FIG. 6B). The sensor chip was regenerated with MgCl₂ from the Human Antibody Capture Kit without any loss of binding capacity. Calculation of association (kₒₙ) and dissociation (k_{off}) rate constants was based on a 1:1 Langmuir binding model. The equilibrium dissociation constant (K_{d}) was calculated from k_{off}/kₒₙ. For epitope mapping studies, each Fab was prepared at 500 nM alone in 1×HBS-EP+ running buffer and then injected in order as indicated in FIG. 5. The results in FIG. 5 show that ERR1-324 is able to bind concurrently with ERR1-Top43, i.e., they do not compete for binding. ERR1-Top43 hinders concurrent binding of ERR+-306, XBR1-402 and ERR1-Top40.

### Example 5. Expression of Purified, Recombinant Strep-Tagged Human ROR1 and Human Twin Strep-Tagged Human ROR2

StrepII-tagged human ROR1-extracellular domain was produced as follows: the nucleotide sequence encoding the extracellular domain of human ROR1 (NP_005003.2) was N-terminally fused to a signal sequence (MNFGLRLIFLVLTLKGVQC) and C-terminally fused with a sequence encoding a peptide comprising a strepII-tag (WSHPQFEK). The entire nucleotide sequences with flanking 5′NotI and 3′HindIII sites were produced by total gene synthesis (GenScript, Piscataway, USA), assembled in the proprietary mammalian expression vector pEvi5 by Evitria AG (Schlieren, Switzerland) and verified by DNA sequencing.

Expression of the proteins was performed in suspension-adapted CHO K1. Supernatants from pools of transfected CHO K1 cells were harvested by centrifugation and sterile filtered (0.2 μm) before FPLC-based affinity purification using StrepTactin columns (IBA GmbH, Goettingen, Germany).

Recombinant human twin strep-tagged ROR2 (NP_004551.2; twin strep sequence WSHPQFEKGGGSGGGSGGSAWSHPQFEKGS) was expressed and purified in-house according to the following protocol: the EBNA expression vector pCB14b-ROR2-ECD-TwinStrep, directing expression of ROR2 extracellular domain (ECD), C-terminally tagged with a TwinStep tag, was transfected into HEK293T using Lipofectamine® LTX with PLUS™ Reagent (Thermo Fisher Scientific, 15388100). Following a 1-day incubation (37° C., 5% CO₂, growth media: Dulbecco's Modified Eagle Medium (DMEM) High Glucose (4.5 g/L) with L-Glutamine with 10% (v/v) Fetal Calf Serum (FCS), 100 IU/mL of Pen-Strep-Fungizone and 2 mM L-glutamine (all Bioconcept)), cells were expanded under selection conditions (2 μg/mL of puromycin (Sigma-Aldrich, P8833-25 mg stock at 2 mg/mL)). Cells were split and further expanded (37° C., 5% CO2); once confluency was reached, tissue culture dishes were coated with 20 μg/ml poly-L-Lysine (Sigma-Aldrich, P1524) for 2 h at 37° C. and washed twice with PBS. Then, cells were trypsinized, washed with PBS and split 1:3 onto poly-L-lysine-coated plates. Again after reaching confluency, cells were washed with PBS followed by with media replacement using production media (DMEM/F-12, Gibco/Thermo Fisher Scientific, 31330-03) supplemented with 1 μg/mL puromycin (Sigma-Aldrich, P8833), 100 IU/mL of Pen-Strep-Fungizone (Bioconcept, 4-02F00-H), 161 μg/mL of N-acetyl-L-cysteine (Sigma-Aldrich, A8199) and 10 μg/mL of L-glutathione reduced (Sigma-Aldrich, G6529). Supernatant, harvested bi-weekly and filtered (0.22 μm) to remove cells, was stored at 4° C. until purification. For purification, filtered supernatant was loaded onto a Streptactin® Superflow® high capacity cartridge (IBA, Gottingen, Germany, 2-1238-001) column; purification and elution was performed according to the manufacturer's protocol on an AEKTA pure (GE Healthcare). Fractions were analyzed for protein purity and integrity by SDS-PAGE. Protein-containing fractions were mixed and subjected to buffer exchange using Amicon filtration units (Millipore, Schaffhausen, Switzerland) to reach a dilution of ≥1:100 in PBS, and then sterile filtered using a low retention filter (0.20 μm, Carl Roth, Karlsruhe, Germany, PA49.1).

### Example 6. Expression of Purified, Recombinant Anti-Human ROR1 and Isotype Control Antibodies

Expression vectors: Antibody variable region coding regions were produced by total gene synthesis (GenScript) using MNFGLRLIFLVLTLKGVQC as leader sequence, and were assembled with human IgH-γ1 and IgL-κ or IgL-λ constant regions, as applicable, in the expression vector pCB14. This vector, a derivative of the episomal mammalian expression vector pCEP4 (Invitrogen), carries the EBV replication origin, encodes the EBV nuclear antigen (EBNA-1) to permit extrachromosomal replication, and contains a puromycin selection marker in place of the original hygromycin B resistance gene.

Expression and purification of ROR1 antibodies: pCB14-based expression vectors were transfected into HEK293T cells using Lipofectamine® LTX Reagent with PLUS™ Reagent (Thermo Fisher Scientific, Reinach, Switzerland, 15388100); following a 1-day incubation (37° C., 5% CO2, growth media: Dulbecco's Modified Eagle Medium (DMEM) High Glucose (4.5 g/L) with L-Glutamine with 10% (v/v) Fetal Calf Serum (FCS), 100 IU/mL of Pen-Strep-Fungizone and 2 mM L-glutamine (all Bioconcept, Allschwil, Switzerland)), cells were expanded under selection conditions (2 μg/mL of puromycin (Sigma-Aldrich, Buchs SG, Switzerland, P8833-25 mg stock at 2 mg/mL)). Cells were split and further expanded (37° C., 5% CO2); once confluency was reached, tissue culture dishes were coated with 20 μg/ml poly-L-Lysine (Sigma-Aldrich, P1524) for 2 h at 37° C. and washed twice with PBS. Then, cells were trypsinized and split 1:3 onto poly-L-lysine-coated plates. Again after reaching confluency, cells were washed with PBS followed by media replacement to production media (DMEM/F-12, Gibco/Thermo Fisher Scientific, 31330-03) supplemented with 1 μg/mL puromycin (Sigma, P8833), 100 IU/mL of Pen-Strep-Fungizone (Bioconcept), 161 μg/mL of N-acetyl-L-cysteine (Sigma-Aldrich, A8199) and 10 μg/mL of L-glutathione reduced (Sigma-Aldrich, G6529). Supernatant, harvested bi-weekly and filtered (0.22 μm) to remove cells, was stored at 4° C. until purification.

For purification, filtered supernatant was loaded onto a PBS-equilibrated Protein A HiTrap column (GE Healthcare, Frankfurt am Main, Germany, 17-0405-01) or a JSR Amsphere™ Protein A column (JSR Life Sciences, Leuven, Belgium, JWT203CE) and washed with PBS; elution was performed using 0.1M glycine (pH 2.5) on an AEKTA pure (GE Healthcare). Fractions were immediately neutralized with 1M Tris-HCl buffer (pH 8.0), and analyzed for protein purity and integrity by SDS-PAGE. Protein-containing fractions were mixed and subjected to buffer exchange using Amicon filtration units (Millipore, Schaffhausen, Switzerland, UFC901008) to reach a dilution of 1:100 in PBS, and then sterile filtered using a low retention filter (0.20 μm, Carl Roth, Karlsruhe, Germany, PA49.1).

Isotype control antibodies were transiently expressed in CHO cells by methods known in the art and recombinant antibodies were purified by standard protein A purification from CHO cell supernatants, as known in the art. The purity and the integrity of the recombinant antibodies were analyzed by SDS-PAGE.

TABLE 2
Protocols and concentrations of anti-hROR1 antibodies of the Examples

| | | | C-Terminal Tags | Final |
|---|---|---|---|---|
| Antibody | | Antibody SEQ ID | (HC: Heavy Chain, | conc. |
| (ref.) | Format | HC/LC | LC: Light Chain) | (mg/mL) |
| XBR1-402 (mAb031) | IgG | HC: SEQ ID NO. 8 | HC: LPETG-Strep | 3.9 |
| | | LC: SEQ ID NO. 9 | LC: G₅SLPETG-Strep | |
| ERR1-301 (mAb027) | IgG | HC: SEQ ID NO. 4 | HC: LPETG-Strep | 3.8 |
| | | LC: SEQ ID NO. 5 | LC: G₅SLPETG-Strep | |
| ERR1-306 (mAb033) | IgG | HC: SEQ ID NO. 6 | HC: LPETG-Strep | 3.0 |
| | | LC: SEQ ID NO. 7 | LC: G₅SLPETG-Strep | |
| ERR1-324 (mAb034) | IgG | HC: SEQ ID NO. 2 | HC: LPETG-Strep | 3.0 |
| | | LC: SEQ ID NO. 3 | LC: G₅SLPETG-Strep | |
| ERR1-403 (mAb035) | IgG | HC: SEQ ID NO. 10 | HC: LPETG-Strep | 2.9 |
| | | LC: SEQ ID NO. 11 | LC: G₅SLPETG-Strep | |
| ERR1-Top43 (mAb036) | IgG | HC: SEQ ID NO. 12 | HC: LPETG-Strep | 3.0 |
| | | LC: SEQ ID NO. 13 | LC: G₅SLPETG-Strep | |
| XBR1-402 scFv | scFv-Ig | SEQ ID NO. 14 | HC: C-terminal | 5.2 |
| (mAb117) | | | LPETG-Strep-GS | |
| ERR1-324-scFv | scFv-Ig | SEQ ID NO. 15 | HC: C-terminal | 4.3 |
| (mAb121) | | | LPETG-Strep-GS | |
| XBR1-402-scFv-324-scFv | scFv-Fc- | Knob: SEQ ID NO. 16 | Knob: LPETG-Strep- | 3.3 |
| (mAb113) | KIH | Hole: SEQ ID NO. 17 | GS | |
| | | | Hole: LPETG-Strep- | |
| | | | GS | |
| ERR1-324-LL-R12 | DvD-Ig | HC: SEQ ID NO. 18 | HC: LPETG- | 1.7 |
| (mAb213) | | LC: SEQ ID NO. 19 | TwinStrep-GS | |
| | | | LC: G₅SLPETG- | |
| | | | TwinStrep-GS | |
| R12 (mAb067) | IgG | HC: SEQ ID NO. 20 | HC: LPETG-Strep | 5.7 |
| | | LC: SEQ ID NO. 21 | LC: G₅SLPETG-Strep | |
| 2A2 (mAb066) | IgG | HC: SEQ ID NO. 22 | HC: LPETG-Strep | 8.0 |
| | | LC: SEQ ID NO. 23 | LC: G₅SLPETG-Strep | |
| Ac10 (mAb046) | IgG | HC: SEQ ID NO. 24 | HC: LPETG-Strep | 7.9 |
| | | LC: SEQ ID NO. 25 | LC: G₅SLPETG-Strep | |
| Trastuzumab (mAb042) | IgG | HC: SEQ ID NO. 26 | HC: LPETG-Strep | 2.7 |
| | | LC: SEQ ID NO. 27 | LC: G₅SLPETG-Strep | |
| scFv-Ig_324_4-2 | scFv-IgG | HC: SEQ ID NO. 67 | HC: LPETG- | 2.7 |
| (mAb351) | | LC: SEQ ID NO. 68 | TwinStrep | |
| | | | LC: G₅SLPETG-Strep | |

### Example 7. mAb ROR1 and ROR2-Binding-Characterization by ELISA

Each well of a 96-well plate was coated with 100 μL of 2 μg/mL strep-tagged human ROR1 or ROR2 (from Example 5) in 0.1 M bicarbonate coating buffer (pH 9.6), and incubated for 12 h at 4° C.

After blocking with 150 μL of 3% (w/v) bovine serum albumin (BSA)/TBS for 1 h at 37° C., the following antibodies were added to a well within each plate at a concentration of 0.5 μg/mL, and serially diluted (dilution factor 4) with 1% (w/v) BSA/TBS, before incubation for 1 h at 37° C.: ERR1-301 (mAb027), XBR1-402 (mAb031), ERR1-306 (mAb033), ERR1-324 (mAb034), ERR1-403 (mAb035) and ERR1-Top43 (mAb036). HRP-conjugated F(ab′)2 anti-human FC-gamma (Jackson Immunoresearch, 109-036-008) was then added at a 1:20′000 dilution, 100 μl per well, and incubated for 1 h at 37° C. prior to detection using an Spark 10M plate reader (Tecan). As shown in FIG. 7, the anti-human ROR1 antibodies bind human ROR1 (panel A) and are not cross-reactive with human ROR2 (panel B).

### Example 8. FACS Staining of Cells for hROR1 Expression

5×10⁵ of each cell type were added per well to 96-well plates. Plates were centrifuged (3 min, 1300 rpm) with re-suspension in buffer (PBS supplemented with 2% (v/v) of FCS). 2A2 (mAb066) was added to each well to reach a concentration of 2 μg/mL. Plates were then incubated on ice for 30 min and washed with 2004 of buffer prior to resuspension in 2004 of buffer supplemented with anti-human IgG antibody (Fc gamma-specific) PE (eBioscience 12-4998-82) at a 1:250 dilution. Following 30 min incubation on ice and one washing, cells were analyzed using a FACSCalibur instrument (BD Biosciences) and data was analyzed using FlowJo analytical software (Tree Star, Ashland, Oreg.).

FIG. 8 shows the FACS analysis data of ROR1-positive human ALL cell lines 697, human triple-negative breast cancer cell lines MDA-MB-468 and HS-578T, human lung cancer cell line A549, human colon cancer cell line HT-29, and ROR1-negative human breast cancer cell line T47D as a negative control.

### Example 9. Conjugation of mAbs with Glycine-Modified Toxins to Form ADCs Using SMAC-Technology™

Sortase A. Recombinant and affinity purified Sortase A enzyme from Staphylococcus aureus was produced in E. coli as disclosed in WO2014140317A1.

Generation of glycine-modified toxins. In order to generate SMAC-Technology™ conjugated ADCs with pentaglycine-modified EDA-anthracycline derivative (G5-PNU) was manufactured by Concortis (FIG. 9). Triglycine-modified EDA-anthracycline derivative (G3-PNU) differs from G5-PNU in that it is modified with only 3 glycine residues instead of 5. The identity and the purity of the pentaglycine-modified and triglycine-modified toxin was confirmed by mass-spectrometry and HPLC. The Gly₅-modified and Gly₃-modified toxin exhibited >95% purity, as gauged by the single peak in the HPLC chromatogram.

Sortase-mediated antibody conjugation. The above-mentioned toxin was conjugated to anti-ROR1 antibodies as per Table 3 by incubating LPETG-tagged mAbs [10 μM] with glycine modified toxin [200 μM] and 3 μM Sortase A in the listed conjugation buffer for 3.5 h at 25° C. The reaction was stopped by passing it through an rProtein A GraviTrap column (BioRad). Bound conjugate was eluted with 5 column volumes of elution buffer (0.1 M glycine pH 2.5, 50 nM NaCl), with 1 column volume fractions collected into tubes containing 25% v/v 1M HEPES pH 8 to neutralise the acid. Protein containing fractions were pooled and formulated in the formulation buffer of Table 3 using a ZebaSpin desalting column.

ADC analytics. DAR was assessed by Reverse Phase Chromatography performed on a Polymer Labs PLRP 2.1 mm×5 cm, 5 μm column run at 1 mL/min/80° C. with a 25 minute linear gradient between 0.05 and 0.1% TFA/H₂O and 0.04 to 0.1% TFA/CH₃CN. Samples were first reduced by incubation with DTT at pH 8.0 at 37° C. for 15 minutes. The DAR determined by Reverse Phase Chromatography is summarized in Table 3 below.

TABLE 3
Analytical summary of ADCs manufactured in this study.

| ADC (ref.) | mAb (ref.) | Toxin | Conjugation Buffer | Formulation Buffer | DAR |
|---|---|---|---|---|---|
| XBR1-402- | XBR1-402 | G5-PNU | 50 mM HEPES (pH 7.5), | PBS without Ca²⁺ and | ND |
| G5-PNU | (mAb031) | | 150 mM NaCl, 5 mM CaCl₂ | Mg²⁺ (Amimed- | |
| (Adc135) | | | | Bioconcept) | |
| ERR1-301- | ERR1-301 | G5-PNU | 50 mM HEPES (pH 7.5), | PBS without Ca²⁺ and | 3.7 |
| G5-PNU | (mAb027) | | 150 mM NaCl, 1 mM CaCl₂ | Mg²⁺ (Amimed- | |
| (Adc200) | | | | Bioconcept) | |
| ERR1-306- | ERR1-306 | G5-PNU | 50 mM HEPES (pH 7.5), | PBS without Ca²⁺ and | 3.7 |
| G5-PNU | (mAb027) | | 150 mM NaCl, 1 mM CaCl₂ | Mg²⁺ (Amimed- | |
| (Adc201) | | | | Bioconcept) | |
| ERR1-324- | ERR1-324 | G5-PNU | 50 mM HEPES (pH 7.5), | PBS without Ca²⁺ and | 3.6 |
| G5-PNU | (mAb034) | | 150 mM NaCl, 1 mM CaCl₂ | Mg²⁺ (Amimed- | |
| (Adc202) | | | | Bioconcept) | |
| ERR1-403- | ERR1-403 | G5-PNU | 50 mM HEPES (pH 7.5), | PBS without Ca²⁺ and | 3.7 |
| G5-PNU | (mAb035) | | 150 mM NaCl, 1 mM CaCl₂ | Mg²⁺ (Amimed- | |
| (Adc203) | | | | Bioconcept) | |
| Top43-G5- | ERR1- | G5-PNU | 50 mM HEPES (pH 7.5), | PBS without Ca²⁺ and | 3.6 |
| PNU | Top43 | | 150 mM NaCl, 1 mM CaCl₂ | Mg²⁺ (Amimed- | |
| (Adc204) | (mAb036) | | | Bioconcept) | |
| ERR1-324- | ERR1- | G5-PNU | 50 mM HEPES (pH 7.5), | 10 mM Succinate pH | ND |
| LL-R12-G5- | 324-LL- | | 150 mM NaCl, 1 mM CaCl₂ | 5.0, 175 mM Sucrose | |
| PNU | R12 | | | 0.02% Tween 20 | |
| (Adc402) | (mAb213) | | | | |
| XBR1-402- | XBR1- | G5-PNU | 50 mM HEPES (pH 7.5), | PBS without Ca²⁺ and | ND |
| scFv-G5- | 402-scFv | | 150 mM NaCl, 1 mM CaCl₂ | Mg²⁺ (Amimed- | |
| PNU | (mAb117) | | | Bioconcept) | |
| (adc251) | | | | | |
| ERR1-324- | ERR1- | G5-PNU | 50 mM HEPES (pH 7.5), | 10 mM Succinate pH | ND |
| scFv-G5- | 324-scFv | | 150 mM NaCl, 1 mM CaCl₂ | 5.0, 175 mM Sucrose, | |
| PNU | (mAb121) | | | 0.02% Tween 20 | |
| (adc252) | | | | | |
| XBR1-402- | XBR1- | G5-PNU | 50 mM HEPES (pH 7.5), | 10 mM Succinate pH | ND |
| scFv-324- | 402-scFv- | | 150 mM NaCl, 1 mM CaCl₂ | 5.0, 175 mM Sucrose, | |
| scFv-G5- | 324-scFv | | | 0.02% Tween 20 | |
| PNU | (mAb113) | | | | |
| (adc249) | | | | | |
| 2A2-G5- | 2A2 | G5-PNU | 50 mM HEPES (pH 7.5), | HEPES buffer saline pH | 3.8 |
| PNU | (mAb066) | | 150 mM NaCl, 1 mM CaCl₂ | 6.8 | |
| (adc165) | | | | | |
| 2A2-G5- | 2A2 | G5-PNU | 50 mM HEPES (pH 7.5), | PBS | 3.5 |
| PNU | (mAb066) | | 150 mM NaCl, 1 mM CaCl₂ | | |
| (adc181) | | | | | |
| R12-G5- | R12 | G5-PNU | 50 mM HEPES (pH 7.5), | 10 mM Succinate pH | 3.8 |
| PNU | (mAb001) | | 150 mM NaCl, 1 mM CaCl₂ | 5.0, 175 mM Sucrose, | |
| (adc050) | | | | 0.02% Tween 20 | |
| R12-G5- | R12 | G5-PNU | 50 mM HEPES (pH 7.5), | PBS without Ca²⁺ and | 3.6 |
| PNU | (mAb067) | | 150 mM NaCl, 1 mM CaCl₂ | Mg²⁺ (Amimed- | |
| (adc263) | | | | Bioconcept) | |
| R12-G5- | R12 | G5-PNU | 25 mM HEPES (pH 7.5), | 10 mM Succinate pH | 3.8 |
| PNU | (mAb067) | | 150 mM NaCl, 1 mM | 5.0, 175 mM Sucrose, | |
| (adc292) | | | CaCl₂), 10% (v/v) glycerol | 0.02% Tween 20 | |
| Tras-G5- | Trastuzumab | G5-PNU | 50 mM HEPES (pH 7.5), | PBS without Ca²⁺ and | 3.6 |
| PNU | (mAb042) | | 150 mM NaCl, 1 mM CaCl₂ | Mg²⁺ (Amimed- | |
| (adc196) | | | | Bioconcept) | |
| Tras-G5- | Trastuzumab | G5-PNU | 50 mM HEPES (pH 7.5), | 25 mM HEPES pH 6.8, | 3.7 |
| PNU | (mAb042) | | 150 mM NaCl, 1 mM CaCl₂ | 15 mM NaCl | |
| (adc286) | | | | | |
| Ac10-G5- | Ac10 | G5-PNU | 50 mM HEPES (pH 7.5), | PBS without Ca²⁺ and | 3.8 |
| PNU | (mAb046) | | 150 mM NaCl, 1 mM CaCl₂ | Mg²⁺ (Amimed- | |
| (adc159) | | | | Bioconcept) | |
| scFv- | scFv- | G3-PNU | 50 mM HEPES (pH 7.5), | PBS | 3.8 |
| Ig_324_4-2- | Ig_324_4- | | 150 mM NaCl, 1 mM CaCl₂ | | |
| G3-PNU | 2 | | | | |
| (adc598) | (mAb351) | | | | |
| Tras-G3- | Trastuzumab | G3-PNU | 50 mM HEPES (pH 7.5), | 10 mM Succinate pH | 3.8 |
| PNU | (mAb302) | | 150 mM NaCl, 1 mM CaCl₂ | 5.0, 175 mM Sucrose, | |
| (adc533) | | | | 0.02% Tween 20 | |
| XBR1-402- | mAb202 | G5-PNU | 50 mM HEPES (pH 7.5), | PBS | 3.9 |
| G5-PNU | | | 150 mM NaCl, 1 mM CaCl₂ | | |
| (adc394) | | | | | |

DAR, drug-to-antibody ratio.
ND, not determined.

From these analyses it can be concluded that the SMAC-Technology™ conjugation has proceeded at high efficiency resulting in overall average DARs in the range of ca. 3.5 to 4.0 for IgG-format anti-ROR1 antibody-toxin combinations.

### Example 10. In Vitro Cytotoxicity of Single and Mixtures of Anti-ROR1 Antibody-Based ADCs on Human 697 B Cell Precursor Leukemia Cells

Cytotoxicity of 50:50 (by weight) mixtures of ERR1-324-G5-PNU with further anti-ROR1 ADCs was investigated using human cell line 697, and compared to the cytotoxicity of the individual ADCs.

For this, 2.5×10⁴ 697 cells per well were plated on 96-well plates (excluding edge wells, which contained water) in 754 RPMI supplemented with 10% by vol. FCS, 100 IU/ml Pen-Strep-Fungizone and 2 mM L-Glutamine and were grown at 37° C. in a humidified incubator at 7.5% CO2 atmosphere. After 1-day incubation, each ADC or ADC mixture was added to respective wells in an amount of 254 of 3.5-fold serial dilutions in growth medium (resulting in final ADC or ADC mixture concentrations from 20 μg/mL to 0.88 ng/ml). After 4 additional days, plates were removed from the incubator and equilibrated to room temperature. After approximately 30 min, 504 was removed from each well, and then 504 of CellTiter-Glo® 2.0 Luminescent Solution (Promega, G9423) was added to each well. After shaking the plates at 750 rpm for 5 min followed by 20 min incubation without shaking, luminescence was measured on a Tecan Infinity F200 plate reader with an integration time of 1 s per well. Curves of luminescence versus ADC concentration (ng/mL) were fitted with Graphpad Prism Software. The IC₅₀ values were determined using the built-in “log(inhibitor) vs. response—Variable slope (four parameters)” IC₅₀ determination function of Prism Software.

TABLE 4
In vitro cell killing of 697 cells by anti-ROR1 ADCs
or ADC mixtures (IC₅₀, ng/mL), NA = Not Available

| ADC | 697 (repeated twice) |
|---|---|
| XBR1-402-G5-PNU (adc135) | 152/124 |
| ERR1-324-G5-PNU (adc202) | 1'580/1'034 |
| XBR1-402-G5-PNU (adc135)/ERR1-324-G5-PNU | 16.8/14 |
| (adc202) mixture | |
| Tras-G5-PNU (adc196) | 6'920/NA |

FIG. 10A shows the dose-repose curves of the in vitro cell killing assays on 697 cells with the ADCs of Table 4. As per the Table and Figure, for the same total dose of ADC, the mixture of ADCs of the invention provides synergistic killing of human 697 B cell precursor leukemia cells that is superior to the individual ADCs and to the isotype control.

TABLE 5
In vitro cell killing of 697 cells by anti-
ROR1 ADCs or ADC mixtures (IC₅₀, ng/mL)

| ADC | 697 |
|---|---|
| R12-G5-PNU (adc050) | 873 |
| ERR1-324-G5-PNU (adc202) | 1'034 |
| R12-G5-PNU (adc050)/ERR1-324-G5-PNU | 37 |
| (adc202) mixture | |

FIG. 10B shows the dose-respose curves of the in vitro cell killing assays on 697 cells with the ADCs of Table 5. For the same total dose of ADC, the mixture of ADCs of the invention provides synergistic killing of human 697 B cell precursor leukemia cells that is superior to the individual ADCs.

TABLE 6
In vitro cell killing of 697 cells by anti-
ROR1 ADCs or ADC mixtures (IC₅₀, ng/mL)

| ADC | 697 |
|---|---|
| 2A2-G5-PNU (adc181) | 125 |
| ERR1-324-G5-PNU (adc202) | 1'034 |
| 2A2-G5-PNU (adc181)/ERR1-324-G5-PNU | 7 |
| (adc202) mixture | |

FIG. 10C shows the dose-response curves of the in vitro cell killing assays on 697 cells with the ADCs of Table 6. For the same total dose of ADC, the mixture of ADCs of the invention provides synergistic killing of human 697 B cell precursor leukemia cells that is superior to the individual ADCs.

TABLE 7
In vitro cell killing of 697 cells by anti-
ROR1 ADCs or ADC mixtures (IC₅₀, ng/mL)

| ADC | 697 |
|---|---|
| ERR1-301-G5-PNU (adc200) | 592 |
| ERR1-324-G5-PNU (adc202) | 1'034 |
| ERR1-301-G5-PNU (adc200)/ERR1-324-G5-PNU | 110 |
| (adc202) mixture | |

FIG. 10D shows the dose-response curves of the in vitro cell killing assays on 697 cells with the ADCs of Table 7. For the same total dose of ADC, the mixture of ADCs of the invention provides synergistic killing of human 697 B cell precursor leukemia cells that is superior to the individual ADCs.

TABLE 8
In vitro cell killing of 697 cells by anti-
ROR1 ADCs or ADC mixtures (IC₅₀, ng/mL)

| ADC | 697 |
|---|---|
| Top43-G5-PNU (adc204) | 314 |
| ERR1-324-G5-PNU (adc202) | 1'034 |
| Top43-G5-PNU (adc204)/ERR1-324-G5-PNU | 9 |
| (adc202) mixture | |

FIG. 10E shows the dose-response curves of the in vitro cell killing assays on 697 cells with the ADCs of Table 8. For the same total dose of ADC, the mixture of ADCs of the invention provides synergistic killing of human 697 B cell precursor leukemia cells that is superior to the individual ADCs.

### Example 11. In Vitro Cytotoxicity of scFv-Fc Format Anti-ROR1 Bi-Epitope Reactive ADCs (BETR-ADCs™) on Human 697 B Cell Precursor Leukemia Cells

Cytotoxicity of anti-ROR1 scFv-Fc-based bi-epitope reactive ADCs (BETR-ADCs™) and ADC mixtures was investigated using human cell line 697. The same protocol as Example 10 was applied to the ADCs of Table 9.

TABLE 9
In vitro cell killing of 697 cells by scFv-Fc-based
anti-ROR1 ADCs or ADC mixtures (IC₅₀, ng/mL)

| ADC | 697 |
|---|---|
| XBR1-402-G5-PNU (scFv-Fc format, adc251) | 456 |
| ERR1-324-G5-PNU (scFv-Fc format, adc252) | 6'190 |
| XBR1-402-G5-PNU (scFv-Fc format, adc251)/ | 193 |
| ERR1-324-G5-PNU (scFv format, adc252) mixture | |
| XBRl-402-p1-ERR1-324-p2-G5-PNU (scFv-Fc bi- | 236 |
| epitopic reactive, adc249) | |

FIG. 11 shows the dose-response curves of the in vitro cell killing assays on 697 cells with the scFv-Fc-based bi-epitope reactive anti-ROR1 ADCs (BETR-ADCs™) and individual anti-ROR1 ADCs of Table 9. As per the above Table and FIG. 11, for the same total dose of ADC, the mixture of the scFv-Fc-based ADCs and the bi-epitope reactive scFv-Fc-based ADC of the invention provide cell killing of human 697 B cell precursor leukemia cells that is superior to the cell killing achieved with individual scFv-Fc-based ADCs.

### Example 12. In Vitro Cytotoxicity of DVD-Ig-Based Anti-ROR1 Bi-Epitope Reactive ADCs (BETR-ADCs™) on 697 Cells

Cytotoxicity of DVD-Ig-based bi-epitope reactive anti-ROR1 ADCs (BETR-ADCs™) and individual anti-ROR1 ADCs was investigated using human cell line 697. The same protocol as Example 10 was applied to the ADCs of Table 10.

TABLE 10
In vitro cell killing of various human cancer cells by anti-ROR1
ADCs and ADC mixtures, as well as an isotype control (IC₅₀, nM)

| ADC/Cell type | 697 |
|---|---|
| hROR1 status | positive |
| ERR1-324-G5-PNU (adc202) | 24 |
| R12-G5-PNU (adc263) | 100 |
| ERR1-324-LL-R12-G5-PNU (adc402) | 1.7 |
| (DVD-Ig format) | |
| Tras-G5-PNU (adc196) | 542 |

FIG. 12 shows the dose-response curves of the in vitro cell killing assay on 697 cells with the ADCs of Table 10. As per the above Table and FIG. 12, for the same total dose of ADC, the bi-epitope reactive anti-ROR1 DVD-Ig-based ADC of the invention shows cell killing of the ROR1 positive human cancer cells that is superior to the cell killing achieved with individual ADCs.

### Example 13. In Vitro Cytotoxicity of Single and Mixtures of Anti-ROR1 Antibody-Based ADCs on Human Colon Cancer HT-29, Breast Cancer MDA-MB-468, Lung Cancer A549, and Breast Cancer HS 578T Cells

Cytotoxicity of 50:50 mixtures of anti-ROR1 ADCs was investigated using human cell lines: HT-29, MDA-MB-468, A549, HS 578T. For this, the following cells per well were plated on 96-well plates (excluding edge wells, which contained water) and were grown at 37° C. in a humidified incubator at 7.5% CO2 atmosphere in growth medium (DMEM supplemented with 10% by vol. FCS, 100 IU/ml Pen-Strep-Fungizone and 2 mM L-Glutamine).

TABLE 11
Cell plating

| Cell type | Cells per well |
|---|---|
| HT-29 | 2.7 × 10⁴ |
| MDA-MD-468 | 6.7 × 10⁴ |
| A549 | 1.3 × 10⁵ |
| HS 578T | 2.7 × 10⁴ |

After 1-day incubation, each ADC or ADC mixture was added to respective wells in an amount of 254 of 3.5-fold serial dilutions in growth medium (resulting in final ADC or ADC mixture concentrations from 20 μg/mL to 0.88 ng/ml). After 4 additional days, plates were removed from the incubator and equilibrated to room temperature. After approximately 30 min, 50 μL was removed from each well, and then 504 of CellTiter-Glo® 2.0 Luminescent Solution (Promega, G9423) was added to each well. After shaking the plates at 750 rpm for 5 min followed by 20 min incubation without shaking, luminescence was measured on a Tecan Infinity F200 plate reader with an integration time of 1 s per well. Curves of luminescence versus ADC concentration (ng/mL) were fitted with Graphpad Prism Software. The IC₅₀ values, determined using the built-in “log(inhibitor) vs. response—Variable slope (four parameters)” IC₅₀ determination function of Prism Software, are reported in Table 12.

TABLE 12
In vitro cell killing of various human cancer cells by anti-ROR1 ADCs
and ADC mixtures, as well as an isotype control (IC₅₀, ng/mL)

| | | MDA- | | HS |
|---|---|---|---|---|
| ADC/Cell type | HT-29 | MD-468 | A549 | 578T |
| hROR1 status | positive | Positive | positive | positive |
| 2A2-G5-PNU (adc165) | 19'759 | 727 | 76'820 | 1'784 |
| XBR1-402-G5-PNU (adc135) | 23'349 | ND | 176'937 | 1'403 |
| R12-G5-PNU (adc292) | 10'267 | 3'960 | ND | 3'498 |
| ERR1-Top43-G5-PNU | 23'125 | 463 | 109'239 | 707 |
| (adc204) | | | | |
| ERR1-324-G5-PNU (adc202)/ | 12'841 | 19 | 3'701 | 156 |
| 2A2-G5-PNU (adc165) mix | | | | |
| ERR1-324-G5-PNU (adc202)/ | 10'538 | 90 | 7'272 | 328 |
| XBR1-402-G5-PNU (adc135) | | | | |
| mix | | | | |
| ERR1-324-G5-PNU (adc202)/ | 7'899 | 102 | 5'229 | 300 |
| R12-G5-PNU (adc292) mix | | | | |
| ERR1-324-G5-PNU (adc202)/ | 14'107 | 14 | 5'596 | 142 |
| ERR1-Top43-G5-PNU | | | | |
| (adc204) mix | | | | |
| Ac10-G5-PNU (adc159), | 18'882 | 3'650 | ND | 8'408 |
| isotype control | | | | |

FIG. 13 shows the dose-response curves of the in vitro cell killing assays on HT-29, MDA-MB-468, A549, HS 578T cells with the ADCs of Table 12, either as single ADCs (panel A) or ADC-mixture (panel B). As per the above Table and FIG. 13, for the same total dose of ADC, the mixture of selected ADCs of the invention provides cell killing of human ROR1 positive 697 cancer cells that is superior to the cell killing of individual ADCs.

### Example 14. In Vitro Cytotoxicity of scFv-IgG-Based Anti-ROR1 Bi-Epitope Reactive ADCs (BETR-ADCs™) on hROR1-Overexpressing EMT-6 Cells

Cell line engineering for ectopic expression of hROR1 in the EMT-6 murine breast cancer cell line: Murine EMT-6 breast cancer cells were cultured in DMEM complete (Dulbecco's Modified Eagle Medium (DMEM) High Glucose (4.5 g/1) with L-Glutamine with 10% (v/v) Fetal Calf Serum (FCS), 100 IU/mL of Pen-Strep-Fungizone and 2 mM L-glutamine (all Bioconcept, Allschwil, Switzerland)) at 37° C. and 5% CO2. Cells were engineered to overexpress ROR1 by transposition as follows: cells were centrifuged (6 min, 1200 rpm, 4° C.) and resuspended in RPMI-1640 media (5×10⁶ cells/mL). 400 μL of this cell suspension was then added to 400 μL of RPMI containing 13.3 μg of transposable vector pPB-PGK-Puro-ROR1, directing co-expression of full-length ROR1 (NP_005003.2) and the puromycin-resistance gene, and 6.6 μg of transposase-containing vector pcDNA3.1_hy_mPB. The DNA/EMT-6 cell mixture was transferred to electroporation cuvettes (0.4 cm-gap, 165-2088, BioRad, Cressier, Switzerland) and electroporated using the Biorad Gene Pulser II with capacitance extender at 300V and 950 μf. Then, cells were incubated for 5-10 min at room-temperature. Following the incubation, cells were centrifuged at 1200 rpm for 6 min, washed once and subsequently resuspended in DMEM complete prior to incubation at 37° C. in a humidified incubator at 5% CO2 atmosphere. One day after electroporation, cell pools stably expressing human ROR1 were selected by adding 3 μg/mL puromycin (Sigma-Aldrich, P8833).

ROR1 expression on selected EMT-6-ROR1 cells was confirmed by flow cytometry (not shown). To isolate ROR1-expressing EMT-6 cell clones, following trypsinization, 10⁶ cells were centrifuged in FACS tubes; obtained pellets were resuspended in buffer (PBS with 2% (v/v) FCS). Cells were then incubated with 2A2 (mAb066, Baskar et al., 2012); 30 min, 4° C., final concentration 2 μg/mL), followed by centrifugation and washing. Cells were then resuspended as previously and incubated with anti-human IgG antibody (Fc gamma-specific) PE (eBioscience, Vienna, Austria, 12-4998-82) with a 1:250 dilution in the dark (30 min, 4° C.), washed once in buffer and kept on ice until FACS sorting.

Using a FACS Aria II, cells were single cell sorted into a 96-well flat-bottom plate containing 200 μL of DMEM complete per well. This plate was incubated at 37° C. and clones were expanded to 6-well plates before analysis of ROR1-expression by flow cytometry as outlined above, using a FACSCalibur instrument (BD Biosciences) and FlowJo analytical software (Tree Star, Ashland, Oreg.) for analysis. FIG. 17A shows the FACS analysis data of clone 14 detected with anti-ROR1 antibody 2A2 (mAb066).

Cytotoxicity. Cytotoxicity of an scFv-Ig-based bi-epitope reactive anti-ROR1 ADC (BETR-ADC′) and individual anti-ROR1 ADCs was investigated using the above engineered EMT-6 cells (clone 14). The same protocol as in Example 10 was applied to the ADCs of Table 13, plating 1000 EMT-6 cells per well.

TABLE 13
In vitro cell killing of human cancer cells by anti-ROR1 ADCs
and ADC mixtures, as well as an isotype control (IC₅₀, nM)

| | hROR1-overexpressing |
|---|---|
| ADC/Cell type | EMT-6 cells |
| ERR1-324-G5-PNU (adc 202) | 2.7 |
| XBR1-402-G5-PNU (adc394) | 1.7 |
| scFv-Ig-324-4-2-G3-PNU (adc598) | 0.9 |
| Tras-G3-PNU (adc533) | 4'980 |

FIG. 17B shows the dose-response curves of the in vitro cell killing assay on hROR1-overexpressing EMT-6 cells with the ADCs of Table 13. As per the above Table and FIG. 17B, for the same total dose of ADC, the bi-epitope reactive anti-ROR1 scFv-Ig-based ADC of the invention shows cell killing of the ROR1 positive human cancer cells that is superior to the cell killing achieved with individual ADCs.

### REFERENCES

WO2016102679
WO2014140317
Baskar et al, Int J Med Sci. 2012; 9(3):193-9
Skerra and Plückthun, Science 2401038-41, 1988.
Balakrishnan et al. (2016) Clin Cancer Res. doi: 10.1158/1078-0432
Rebagay et al. (2012) Frontiers Oncol. 2(34):1-8; doi 10.3389
Shatz et al., mAbs 5:6, 872-881 (2013).
Lefranc M P et al., 2015 Nucleic Acids Res. January; 43: D413-22. doi: 10.1093/nar/gku1056
Ward et al., Nature 341:544-546, 1989
Bird et al., Science 242: 423-426, 1988
Huston et al., Proc. Natl. Acad. Sci. USA 85:5879-5883, 1988
Reiter et al., Int. J. Cancer 67:113-23, 1996
Cai and Garen, Proc. Natl. Acad. Sci. USA 93:6280-85, 1996.
Dumoulin et al., Nat. Struct. Biol. 11:500-515, 2002
Ghahroudi et al., FEBS Letters 414:521-526, 1997
Bond et al J. Mol. Biol. 332643-55, 2003.
Chen et al., PNAS 2011; 108(28); 11399-11404.
Dorr B M et al., PNAS 2014; 111, 13343-8.
Beerli et al. (2015) Plos One 10, e131177
Quintieri et al., Clin. Cancer Res 11:1608-1617 (2005)
Cai and Garen, Proc. Natl. Acad. Sci. USA 93:6280-85, 1996
Harlow & Lane, Using Antibodies, A Laboratory Manual, Cold Spring Harbor Laboratory Press, Cold Spring Harbor, N.Y., 1998.
Shinkai et al. (1992) Cell 68:855-67
Popkov et al., J. Mol. Biol. 325:325-335, 2003.
Yang et al Plos One 6:e21018, 2011.
McCartney-Francis et al., Proc. Natl. Acad. Sci. USA 81:1794-1798,
Hofer et al., J Immunol Meth 318:75-87, 2007
Rader, Methods Mol Biol 525:101-128, xiv, 2009.
Yang and Rader, Methods Mol Biol 901:209-232, 2012
Garen, Proc. Natl. Acad. Sci. USA 93:6280-85, 1996
Kwong and Rader, Curr Protoc Protein Sci Chapter 6:Unit 6 10, 2009

### SEQUENCES

The following sequences form part of the disclosure of the present application. A WIPO ST 25 compatible electronic sequence listing is provided with this application, too. For the avoidance of doubt, if discrepancies exist between the sequences in the following table and the electronic sequence listing, the sequences in this table shall be deemed to be the correct ones.

| No | Qualifier | Sequence |
|---|---|---|
| 1 | hROR1 | QETELSVSAELVPTSSWNISSELNKDSYLTLDEPMNNITTSLGQTAELHCKV |
| | extracellular | SGNPPPTIRWEKNDAPVVQEPRRLSERSTIYGSRLRIRNLDTTDTGYFQCVA |
| | domain amino | TNGKEVVSSTGVLEVKFGPPPTASPGYSDEYEEDGFCQPYRGIACARFIGNR |
| | acid sequence | TVYMESLHMQGEIENQITAAFTMIGTSSHLSDKCSQFAIPSLCHYAFPYCDE |
| | | TSSVPKPRDLCRDECEILENVLCQTEYIFARSNPMILMRLKLPNCEDLPQPE |
| | | SPEAANCIRIGIPMADPINKNHKCYNSTGVDYRGTVSVTKSGRQCQPWNSQY |
| | | PHTHTFTALREPELNGGHSYCRNPGNQKEAPWCFTLDENEKSDLCDIPACDS |
| | | KDSKEKNKMEILY |
| 2 | ERR1-324HC | QSLEESGGGLVQPGESLTLTCTVSGFSLSRNGMTWVRQAPGKGLEWIGIITS |
| | amino acid | SGDKYYATWAKGRETISKTSSTTVDLKMTSLTTEDTATYFCARGTVSSDIWG |
| | sequence | PGTLVTISSASTKGPSVFPLAPSSKSTSGGTAALGCLVKDYFPEPVTVSWNS |
| | | GALTSGVHTFPAVLQSSGLYSLSSVVTVPSSSLGTQTYICNVNHKPSNTKVD |
| | | KKVEPKSCDKTHTCPPCPAPELLGGPSVFLEPPKPKDTLMISRTPEVTCVVV |
| | | DVSHEDPEVKFNWYVDGVEVHNAKTKPREEQYNSTYRVVSVLTVLHQDWLNG |
| | | KEYKCKVSNKALPAPIEKTISKAKGQPREPQVYTLPPSRDELTKNQVSLTCL |
| | | VKGFYPSDIAVEWESNGQPENNYKTTPPVLDSDGSFELYSKLTVDKSRWQQG |
| | | NVFSCSVMHEALHNHYTQKSLSLSPGK |
| 3 | ERR1-324LC | ELVLTQTPSPVSAAVGGTVTINCQASQSVYGNNELAWYQQKPGQPPKLLIYR |
| | amino acid | ASILTSGVPSRFKGSGSGTQFTLTISNVQREDAATYYCLGGYVSQSYRAAFG |
| | sequence | GGTELEILRTVAAPSVFIFPPSDEQLKSGTASVVCLLNNFYPREAKVQWKVD |
| | | NALQSGNSQESVTEQDSKDSTYSLSSTLTLSKADYEKHKVYACEVTHQGLSS |
| | | PVTKSFNRGEC |
| 4 | ERR1-301HC | QEQLEESGGGLVTPGGSLTLTCTASGETISTYHMSWVRQAPGKGLEWIGSTY |
| | amino acid | AGSGSTYYASWVNGRETISSNTTQNTVSLQMNSLTVADTATYFCARDHPSYG |
| | sequence | MDLWGPGTLVTVSSASTKGPSVFPLAPSSKSTSGGTAALGCLVKDYFPEPVT |
| | | VSWNSGALTSGVHTFPAVLQSSGLYSLSSVVTVPSSSLGTQTYICNVNHKPS |
| | | NTKVDKKVEPKSCDKTHTCPPCPAPELLGGPSVFLEPPKPKDTLMISRTPEV |
| | | TCVVVDVSHEDPEVKFNWYVDGVEVHNAKTKPREEQYNSTYRVVSVLTVLHQ |
| | | DWLNGKEYKCKVSNKALPAPIEKTISKAKGQPREPQVYTLPPSRDELTKNQV |
| | | SLTCLVKGFYPSDIAVEWESNGQPENNYKTTPPVLDSDGSFELYSKLTVDKS |
| | | RWQQGNVFSCSVMHEALHNHYTQKSLSLSPGK |
| 5 | ERR1-301LC | SYELTQLPSVSVSLGQTARITCGGNSIGSKAVNWYQQKPGLAPGLLIYDDDE |
| | amino acid | RPSGVPDRESASNSGDTATLTISGAQAEDEADYYCQLWDSSAVAYVEGGGTQ |
| | sequence | LTVTGQPKAAPSVTLEPPSSEELQANKATLVCLISDFYPGAVTVAWKADSSP |
| | | VKAGVETTTPSKQSNNKYAASSYLSLTPEQWKSHKSYSCQVTHEGSTVEKTV |
| | | APTECS |
| 6 | ERR1-306HC | QEQLKESGGGLVQPGGSLKLSCKASGFDLSNYGVSWVRQAPGKGLEWIGYID |
| | amino acid | PTEDYTYYASWVNGRESISRENTQNTVSLQINSLTPADTATYFCARWVYGVD |
| | sequence | DYGDGNWLDLWGQGTLVTISSASTKGPSVFPLAPSSKSTSGGTAALGCLVKD |
| | | YEPEPVTVSWNSGALTSGVHTFPAVLQSSGLYSLSSVVTVPSSSLGTQTYIC |
| | | NVNHKPSNTKVDKKVEPKSCDKTHTCPPCPAPELLGGPSVFLFPPKPKDTLM |
| | | ISRTPEVTCVVVDVSHEDPEVKFNWYVDGVEVHNAKTKPREEQYNSTYRVVS |
| | | VLTVLHQDWLNGKEYKCKVSNKALPAPIEKTISKAKGQPREPQVYTLPPSRD |
| | | ELTKNQVSLTCLVKGFYPSDIAVEWESNGQPENNYKTTPPVLDSDGSFELYS |
| | | KLTVDKSRWQQGNVFSCSVMHEALHNHYTQKSLSLSPGK |
| 7 | ERR1-306LC | QFVLTQSPSVSAALGASAKLTCTLSSAHKTYTIDWYQQQQGEAPRYLMELKS |
| | amino acid | DGSYTKGTGVPDRFSGSSSGADRYLIIPSVQADDEADYYCGTDYSGGYVFGG |
| | sequence | GTQLTVTGQPKAAPSVTLEPPSSEELQANKATLVCLISDFYPGAVTVAWKAD |
| | | SSPVKAGVETTTPSKQSNNKYAASSYLSLTPEQWKSHKSYSCQVTHEGSTVE |
| | | KTVAPTECS |
| 8 | XBR1-402 HC | QEQQKESGGGLEKPTDTLTLTCTASGEDISSYYMSWVRQAPGNGLEWIGAIG |
| | amino acid | ISGNAYYASWAKSRSTITRNTNLNTVTLKMTSLTAADTATYFCARDHPTYGM |
| | sequence | DLWGPGTLVTVSSASTKGPSVFPLAPSSKSTSGGTAALGCLVKDYFPEPVTV |
| | | SWNSGALTSGVHTFPAVLQSSGLYSLSSVVTVPSSSLGTQTYICNVNHKPSN |
| | | TKVDKKVEPKSCDKTHTCPPCPAPELLGGPSVFLEPPKPKDTLMISRTPEVT |
| | | CVVVDVSHEDPEVKFNWYVDGVEVHNAKTKPREEQYNSTYRVVSVLTVLHQD |
| | | WLNGKEYKCKVSNKALPAPIEKTISKAKGQPREPQVYTLPPSRDELTKNQVS |
| | | LTCLVKGFYPSDIAVEWESNGQPENNYKTTPPVLDSDGSFELYSKLTVDKSR |
| | | WQQGNVFSCSVMHEALHNHYTQKSLSLSPGK |
| 9 | XBR1-402 LC | SYELTQLPSVSVSLGQTARITCEGNNIGSKAVHWYQQKPGLAPGLLIYDDDE |
| | amino acid | RPSGVPDRFSGSNSGDTATLTISGAQAGDEADYYCQVWDSSAYVEGGGTQLT |
| | sequence | VTGQPKAAPSVTLEPPSSEELQANKATLVCLISDFYPGAVTVAWKADSSPVK |
| | | AGVETTTPSKQSNNKYAASSYLSLTPEQWKSHKSYSCQVTHEGSTVEKTVAP |
| | | TECS |
| 10 | ERR1-403HC | QEQLKESGRGLVQPGGSLKLSCKASGEDFSGWYMTWVRQAPGKGLEWIGTIG |
| | amino acid | TTKGRTYYASWVNGRETISSDNAQNTVDLQMNSLTAADRATYFCVRGSDYFD |
| | sequence | LWGPGTLVTISSASTKGPSVFPLAPSSKSTSGGTAALGCLVKDYFPEPVTVS |
| | | WNSGALTSGVHTFPAVLQSSGLYSLSSVVTVPSSSLGTQTYICNVNHKPSNT |
| | | KVDKKVEPKSCDKTHTCPPCPAPELLGGPSVFLEPPKPKDTLMISRTPEVTC |
| | | VVVDVSHEDPEVKFNWYVDGVEVHNAKTKPREEQYNSTYRVVSVLTVLHQDW |
| | | LNGKEYKCKVSNKALPAPIEKTISKAKGQPREPQVYTLPPSRDELTKNQVSL |
| | | TCLVKGFYPSDIAVEWESNGQPENNYKTTPPVLDSDGSFELYSKLTVDKSRW |
| | | QQGNVFSCSVMHEALHNHYTQKSLSLSPGK |
| 11 | ERR1-403LC | SYELTQLPSVSVSLGQTARITCGGNSIGSKAVNWYQQKPGLAPGLLIYDDDE |
| | amino acid | RPSGVPARFSGSNSGDTATLTISGAQAGDEADYYCQLWDSSAGAYVEGGGTQ |
| | sequence | LTVTGQPKAAPSVTLEPPSSEELQANKATLVCLISDFYPGAVTVAWKADSSP |
| | | VKAGVETTTPSKQSNNKYAASSYLSLTPEQWKSHKSYSCQVTHEGSTVEKTV |
| | | APTECS |
| 12 | Top43HC amino | QSLEESGGRLVTPGTPLTLTCTVSGESLSSYWMSWVRQAPGKGLEWIGAIYG |
| | acid sequence | SGNTYYASWAKGRETISKTSTTVDLKITSPTTEDTATYFCARDVHSTATDLW |
| | | GPGTLVTISSASTKGPSVFPLAPSSKSTSGGTAALGCLVKDYFPEPVTVSWN |
| | | SGALTSGVHTFPAVLQSSGLYSLSSVVTVPSSSLGTQTYICNVNHKPSNTKV |
| | | DKKVEPKSCDKTHTCPPCPAPELLGGPSVFLEPPKPKDTLMISRTPEVTCVV |
| | | VDVSHEDPEVKFNWYVDGVEVHNAKTKPREEQYNSTYRVVSVLTVLHQDWLN |
| | | GKEYKCKVSNKALPAPIEKTISKAKGQPREPQVYTLPPSRDELTKNQVSLTC |
| | | LVKGFYPSDIAVEWESNGQPENNYKTTPPVLDSDGSFELYSKLTVDKSRWQQ |
| | | GNVFSCSVMHEALHNHYTQKSLSLSPGK |
| 13 | Top43LC amino | SYELTQLPSVSVSLGQTARITCGGNNIGSKAVNWYQQKPGLAPGLLIYNDDE |
| | acid sequence | RPSGVPDRFSGSNSGDTATLTISGAQAGDEADYYCQLWDSSAGAYVFGGGTQ |
| | | LTVTGQPKAAPSVTLFPPSSEELQANKATLVCLISDFYPGAVTVAWKADSSP |
| | | VKAGVETTTPSKQSNNKYAASSYLSLTPEQWKSHKSYSCQVTHEGSTVEKTV |
| | | APTECS |
| 14 | XBR1-402 scFv | SYELTQLPSVSVSLGQTARITCEGNNIGSKAVHWYQQKPGLAPGLLIYDDDE |
| | HC & LC amino | RPSGVPDRFSGSNSGDTATLTISGAQAGDEADYYCQVWDSSAYVFGGGTQLT |
| | acid sequence | VTGGGGSGGGGSGGGGSQEQQKESGGGLFKPTDTLTLTCTASGFDISSYYMS |
| | | WVRQAPGNGLEWIGAIGISGNAYYASWAKSRSTITRNTNLNTVTLKMTSLTA |
| | | ADTATYFCARDHPTYGMDLWGPGTLVTVSSEPKSSDKTHTCPPCPAPELLGG |
| | | PSVFLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPAPIEKTISKAKG |
| | | QPREPQVYTLPPSREEMTKNQVSLTCLVKGFYPSDIAVEWESNGQPENNYKT |
| | | TPPVLDSDGSFFLYSKLTVDKSRWQQGNVFSCSVMHEALHNHYTQKSLSLSP |
| | | GK |
| 15 | ERR1-324-scFv | ELVLTQTPSPVSAAVGGTVTINCQASQSVYGNNELAWYQQKPGQPPKLLTYR |
| | HC & LC amino | ASILTSGVPSRFKGSGSGTQFTLTISNVQREDAATYYCLGGYVSQSYRAAFG |
| | acid sequence | GGTELEILGGGGSGGGGSGGGGSQSLEESGGGLVQPGESLTLTCTVSGFSLS |
| | | RNGMTWVRQAPGKGLEWIGIITSSGDKYYATWAKGRFTISKTSSTTVDLKMT |
| | | SLTTEDTATYFCARGTVSSDIWGPGTLVTISSEPKSSDKTHTCPPCPAPELL |
| | | GGPSVFLFPPKPKDTLMISRTPEVTCVVVDVSHEDPEVKFNWYVDGVEVHNA |
| | | KTKPREEQYNSTYRVVSVLTVLHQDWLNGKEYKCKVSNKALPAPIEKTISKA |
| | | KGQPREPQVYTLPPSREEMTKNQVSLTCLVKGFYPSDIAVEWESNGQPENNY |
| | | KTTPPVLDSDGSFFLYSKLTVDKSRWQQGNVFSCSVMHEALHNHYTQKSLSL |
| | | SPGK |
| 16 | scFv-402-Fc-KIH- | SYELTQLPSVSVSLGQTARITcEGNNIGsKAVHWYQQKPGLAPGLLIYDDDE |
| | p1 (Knob) | RPSGVPDRFSGSNSGDTATLTISGAQAGDEADYYCQVWDSSAYVFGGGTQLT |
| | | VTGGGGSGGGGSGGGGSQEQQKESGGGLFKPTDTLTLTCTASGFDISSYYMS |
| | | WVRQAPGNGLEWIGAIGISGNAYYASWAKSRSTITRNTNLNTVTLKMTSLTA |
| | | ADTATYFCARDHPTYGMDLWGPGTLVTVSSEPKSSDKTHTCPPCPAPELLGG |
| | | PSVFLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPAPIEKTISKAKG |
| | | QPREPQVYTLPPSREEMTKNQVSLSCAVKGFYPSDIAVEWESNGQPENNYKT |
| | | TPPVLDSDGSFFLVSKLTVDKSRWQQGNVFSCSVMHEALHNHYTQKSLSLSP |
| | | GK |
| 17 | scFv-ERR1-324- | ELVLTQTPSPVSAAVGGTVTINCQASQSVYGNNELAWYQQKPGQPPKLLTYR |
| | Fc-KIH-p2 (Hole) | ASILTSGVPSRFKGSGSGTQFTLTISNVQREDAATYYCLGGYVSQSYRAAFG |
| | | GGTELEILGGGGSGGGGSGGGGSQSLEESGGGLVQPGESLTLTCTVSGFSLS |
| | | RNGMTWVRQAPGKGLEWIGIITSSGDKYYATWAKGRFTISKTSSTTVDLKMT |
| | | SLTTEDTATYFCARGTVSSDIWGPGTLVTISSEPKSSDKTHTCPPCPAPELL |
| | | GGPSVFLFPPKPKDTLMISRTPEVTCVVVDVSHEDPEVKFNWYVDGVEVHNA |
| | | KTKPREEQYNSTYRVVSVLTVLHQDWLNGKEYKCKVSNKALPAPIEKTISKA |
| | | KGQPREPQVYTLPPSREEMTKNQVSLWCLVKGFYPSDIAVEWESNGQPENNY |
| | | KTTPPVLDSDGSFFLYSKLTVDKSRWQQGNVFSCSVMHEALHNHYTQKSLSL |
| | | SPGK |
| 18 | 324-LL-R12 HC | QSLEESGGGLVQPGESLTLTCTVSGESLSRNGMTWVRQAPGKGLEWIGIITS |
| | amino acid | SGDKYYATWAKGRETISKTSSTTVDLKMTSLTTEDTATYFCARGTVSSDIWG |
| | sequence | PGTLVTISSASTKGPSVFPLAPQEQLVESGGRLVTPGGSLTLSCKASGEDFS |
| | | AYYMSWVRQAPGKGLEWIATTYPSSGKTYYATWVNGRETISSDNAQNTVDLQ |
| | | MNSLTAADRATYFCARDSYADDGALFNIWGPGTLVTISSASTKGPSVFPLAP |
| | | SSKSTSGGTAALGCLVKDYFPEPVTVSWNSGALTSGVHTFPAVLQSSGLYSL |
| | | SSVVTVPSSSLGTQTYICNVNHKPSNTKVDKKVEPKSCDKTHTCPPCPAPEL |
| | | LGGPSVFLEPPKPKDTLMISRTPEVTCVVVDVSHEDPEVKFNWYVDGVEVHN |
| | | AKTKPREEQYNSTYRVVSVLTVLHQDWLNGKEYKCKVSNKALPAPIEKTISK |
| | | AKGQPREPQVYTLPPSRDELTKNQVSLTCLVKGFYPSDIAVEWESNGQPENN |
| | | YKTTPPVLDSDGSFFLYSKLTVDKSRWQQGNVFSCSVMHEALHNHYTQKSLS |
| | | LSPGK |
| 19 | 324-LL-R12 LC | ELVLTQTPSPVSAAVGGTVTINCQASQSVYGNNELAWYQQKPGQPPKLLIYR |
| | amino acid | ASILTSGVPSRFKGSGSGTQFTLTISNVQREDAATYYCLGGYVSQSYRAAFG |
| | sequence | GGTELEILRTVAAPSVFIFPPELVLTQSPSVSAALGSPAKITCTLSSAHKTD |
| | | TIDWYQQLQGEAPRYLMQVQSDGSYTKRPGVPDRFSGSSSGADRYLIIPSVQ |
| | | ADDEADYYCGADYIGGYVEGGGTQLTVTGQPKAAPSVTLEPPSSEELQANKA |
| | | TLVCLISDFYPGAVTVAWKADSSPVKAGVETTTPSKQSNNKYAASSYLSLTP |
| | | EQWKSHKSYSCQVTHEGSTVEKTVAPTECS |
| 20 | R12 HC amino | QEQLVESGGRLVTPGGSLTLSCKASGEDFSAYYMSWVRQAPGKGLEWIATIY |
| | acid sequence | PSSGKTYYATWVNGRETISSDNAQNTVDLQMNSLTAADRATYFCARDSYADD |
| | | GALFNIWGPGTLVTISSASTKGPSVFPLAPSSKSTSGGTAALGCLVKDYFPE |
| | | PVTVSWNSGALTSGVHTFPAVLQSSGLYSLSSVVTVPSSSLGTQTYICNVNH |
| | | KPSNTKVDKKVEPKSCDKTHTCPPCPAPELLGGPSVFLEPPKPKDTLMISRT |
| | | PEVTCVVVDVSHEDPEVKFNWYVDGVEVHNAKTKPREEQYNSTYRVVSVLTV |
| | | LHQDWLNGKEYKCKVSNKALPAPIEKTISKAKGQPREPQVYTLPPSRDELTK |
| | | NQVSLTCLVKGFYPSDIAVEWESNGQPENNYKTTPPVLDSDGSFELYSKLTV |
| | | DKSRWQQGNVFSCSVMHEALHNHYTQKSLSLSPGK |
| 21 | R12 LC amino | ELVLTQSPSVSAALGSPAKITCTLSSAHKTDTIDWYQQLQGEAPRYLMQVQS |
| | acid sequence | DGSYTKRPGVPDRFSGSSSGADRYLIIPSVQADDEADYYCGADYIGGYVEGG |
| | | GTQLTVTGQPKAAPSVTLEPPSSEELQANKATLVCLISDFYPGAVTVAWKAD |
| | | SSPVKAGVETTTPSKQSNNKYAASSYLSLTPEQWKSHKSYSCQVTHEGSTVE |
| | | KTVAPTECS |
| 22 | 2A2 HC amino | QVQLQQSGAELVRPGASVTLSCKASGYTFSDYEMHWVIQTPVHGLEWIGAID |
| | acid sequence | PETGGTAYNQKFKGKAILTADKSSSTAYMELRSLTSEDSAVYYCTGYYDYDS |
| | | FTYWGQGTLVTVSAASTKGPSVFPLAPSSKSTSGGTAALGCLVKDYFPEPVT |
| | | VSWNSGALTSGVHTFPAVLQSSGLYSLSSVVTVPSSSLGTQTYICNVNHKPS |
| | | NTKVDKKVEPKSCDKTHTCPPCPAPELLGGPSVFLEPPKPKDTLMISRTPEV |
| | | TCVVVDVSHEDPEVKFNWYVDGVEVHNAKTKPREEQYNSTYRVVSVLTVLHQ |
| | | DWLNGKEYKCKVSNKALPAPIEKTISKAKGQPREPQVYTLPPSRDELTKNQV |
| | | SLTCLVKGFYPSDIAVEWESNGQPENNYKTTPPVLDSDGSFELYSKLTVDKS |
| | | RWQQGNVFSCSVMHEALHNHYTQKSLSLSPGK |
| 23 | 2A2 LC amino | DIVMTQSQKIMSTTVGDRVSITCKASQNVDAAVAWYQQKPGQSPKLLIYSAS |
| | acid sequence | NRYTGVPDRFTGSGSGTDFTLTISNMQSEDLADYFCQQYDIYPYTEGGGTKL |
| | | EIKRTVAAPSVFIFPPSDEQLKSGTASVVCLLNNFYPREAKVQWKVDNALQS |
| | | GNSQESVTEQDSKDSTYSLSSTLTLSKADYEKHKVYACEVTHQGLSSPVTKS |
| | | FNRGEC |
| 24 | Ac10 HC amino | QIQLQQSGPEVVKPGASVKISCKASGYTFTDYYITWVKQKPGQGLEWIGWIY |
| | acid sequence | PGSGNTKYNEKFKGKATLTVDTSSSTAFMQLSSLTSEDTAVYFCANYGNYWF |
| | | AYWGQGTQVTVSAASTKGPSVFPLAPSSKSTSGGTAALGCLVKDYFPEPVTV |
| | | SWNSGALTSGVHTFPAVLQSSGLYSLSSVVTVPSSSLGTQTYICNVNHKPSN |
| | | TKVDKKVEPKSCDKTHTCPPCPAPELLGGPSVFLEPPKPKDTLMISRTPEVT |
| | | CVVVDVSHEDPEVKFNWYVDGVEVHNAKTKPREEQYNSTYRVVSVLTVLHQD |
| | | WLNGKEYKCKVSNKALPAPIEKTISKAKGQPREPQVYTLPPSRDELTKNQVS |
| | | LTCLVKGFYPSDIAVEWESNGQPENNYKTTPPVLDSDGSFELYSKLTVDKSR |
| | | WQQGNVFSCSVMHEALHNHYTQKSLSLSPGK |
| 25 | Ac10 LC amino | DIVLTQSPASLAVSLGQRATISCKAsQsvDEDGDSYMNWYQQKPGQPPKVLI |
| | acid sequence | YAASNLESGIPARFSGSGSGTDFTLNIHPVEEEDAATYYCQQSNEDPWTEGG |
| | | GTKLEIKRTVAAPSVFIFPPSDEQLKSGTASVVCLLNNEYPREAKVQWKVDN |
| | | ALQSGNSQESVTEQDSKDSTYSLSSTLTLSKADYEKHKVYACEVTHQGLSSP |
| | | VTKSFNRGEC |
| 26 | pCEP4-hROR1-F | ATCCTGTTTCTCGTAGCTGCTGCAACTGGAGCACACTCCGCCCGGGGCGCCG |
| | | CCGCCCAG |
| 27 | pCEP4-hROR1- | CCACTCGATCTTCTGGGCCTCGAAGATGTCGTTCAGGCCCTCCATCTTGTTC |
| | Avi-tag-R | TTCTCCTT |
| 28 | pCEP4-signal-F- | GCTGGGTACCGGCGCGCCACCATGGACTGGACTTGGAGAATCCTGTTTCTCG |
| | KpnI | TAGCTGCT |
| 29 | pCEP4-6HIS-R- | GCCGGCCTCGAGTCAGTGATGGTGATGGTGGTGCTCGTGCCACTCGATCTTC |
| | XhoI | TGGGCCTC |
| 30 | hROR1-His_R | CGGCCTCGAGTCAGTGATGGTGATGGTGGTGCTCCATCTTGTTCTTCTCCTT |
| 31 | SP-hROR2_F | GCTGGGTACCGGCGCGCCACCATGGACTGGACTTGGAGAATCCTGTTTCTCG |
| | | TAGCTGCTGCAACTGGAGCACACTCCGAAGTGGAGGTTCTGGATCCG |
| 32 | hROR2-His_R | CGGCCTCGAGTCAGTGATGGTGATGGTGGTGCCCCATCTTGCTGCTGTCTCG |
| 33 | XBR1-402_VH_F | GAGGAGGAGCTCACTCTCAGGAGCAGCAGAAGGAGTCCGGG |
| 34 | XBR1-402_VH_R | CGATGGGCCCTTGGTGGAGGCTGAAGAGACGGTGACGAGGGTCCCTGGCCCC |
| | | CAGAGGTC |
| 35 | XBR1-402_λ_F | GAGAAGCTTGTTGCTCTGGATCTCTGGTGCCTACGGGTCCTATGAGCTGACA |
| | | CAGCTGCC |
| 36 | LEAD-B | GGCCATGGCTGGTTGGGCAGC |
| 37 | KpnI/AscI-Signal | GGTACCGGCGCGCCACCATGGACTGGACTTGGAGAATCCTGTTTCTCGTAGC |
| | | TGCTGCAA |
| 38 | CH1-internal/overlap-R | GCCGCTGGTCAGGGCTCCTG |
| 39 | CH1-internal/overlap-F | CAGGAGCCCTGACCAGCGGC |
| 40 | HC-CH3-R-XhoI | GGCCTCGAGTCATTTACCCGGAGACAGGGA |
| 41 | ERR1-324 HC-F | TTTCTCGTAGCTGCTGCAACTGGAGCACACTCC |
| | | CAGTCGCTGGAGGAGTCCGGG |
| 42 | ERR1-TOP43 HC-F | TTTCTCGTAGCTGCTGCAACTGGAGCACACTCC |
| | | CAGTCGTTGGAGGAGTCCGGG |
| 43 | ERR1-TOP54 HC-F | TTTCTCGTAGCTGCTGCAACTGGAGCACACTCC |
| | | CAGTCGTTGGAGGAGTCCGGG |
| 44 | VH-CH1-R-EheI | GGAGGGCGCCAGGGGGAAGACCGATGGGCCCTTGGT |
| 45 | ERR1-TOP43 LC-F | TTTCTCGTAGCTGCTGCAACTGGAGCACACTCC |
| | | TCCTATGAGCTGACACAGCTG |
| 46 | ERR1-TOP54 LC-F | TTTCTCGTAGCTGCTGCAACTGGAGCACACTCC |
| | | TCCTATGAGCTGACACAGCTG |
| 47 | ERR1-324 KC-F | TTTCTCGTAGCTGCTGCAACTGGAGCACACTCC |
| | | GAGCTCGTGCTGACCCAGACT |
| 48 | LC-R-XhoI | GGCCTCGAGTTATGAACATTCTGTAGGGGC |
| 49 | KC-R-XhoI | GGCCTCGAGTTAACACTCTCCCCTGTTGAA |
| 50 | SP-hROR1_F | GCTGGGTACCGGCGCGCCACCATGGACTGGACTTGGAGAATCCTGTTTCTCG |
| | | TAGCTGCTGCAACTGGAGCACACTCCGCCCGGGGCGCCGCCGCCCAG |
| 51 | Trastuzumab HC | EVQLVESGGGLVQPGGSLRLSCAASGFNIKDTYIHWVRQAPGKGLEWVARIY |
| | amino acid | PTNGYTRYADSVKGRFTISADTSKNTAYLQMNSLRAEDTAVYYCSRWGGDGF |
| | sequence | YAMDYWGQGTLVTVSSASTKGPSVFPLAPSSKSTSGGTAALGCLVKDYFPEP |
| | | VTVSWNSGALTSGVHTFPAVLQSSGLYSLSSVVTVPSSSLGTQTYICNVNHK |
| | | PSNTKVDKKVEPKSCDKTHTCPPCPAPELLGGPSVFLFPPKPKDTLMISRTP |
| | | EVTCVVVDVSHEDPEVKFNWYVDGVEVHNAKTKPREEQYNSTYRVVSVLTVL |
| | | HQDWLNGKEYKCKVSNKALPAPIEKTISKAKGQPREPQVYTLPPSRDELTKN |
| | | QVSLTCLVKGFYPSDIAVEWESNGQPENNYKTTPPVLDSDGSFFLYSKLTVD |
| | | KSRWQQGNVFSCSVMHEALHNHYTQKSLSLSPGK |
| 52 | Trastuzumab LC | DIQMTQSPSSLSASVGDRVTITCRASQDVNTAVAWYQQKPGKAPKLLIYSAS |
| | amino acid | FLYSGVPSRFSGSRSGTDFTLTISSLQPEDFATYYCQQHYTTPPTFGQGTKV |
| | sequence | EIKRTVAAPSVFIFPPSDEQLKSGTASVVCLLNNFYPREAKVQWKVDNALQS |
| | | GNSQESVTEQDSKDSTYSLSSTLTLSKADYEKHKVYACEVTHQGLSSPVTKS |
| | | FNRGEC |
| 53 | Staphylococcus aureus sortase | -LPXTG |
| | A recognition sequence, with | |
| | X being any amino acid | |
| 54 | Staphylococcus aureus sortase | -LPXAG |
| | A recognition sequence, with | |
| | X being any amino acid | |
| 55 | recognition sequence for | -LPXSG |
| | Staphylococcus aureus sortase | |
| | A or engineered sortase A 4S-9 | |
| | from Staphylococcus aureus, | |
| | with X being any amino acid | |
| 56 | recognition sequence for | -LAXTG |
| | engineered sortase A 2A-9 from | |
| | Staphylococcus aureus, with | |
| | X being any amino acid | |
| 57 | Streptococcus pyogenes sortase | -LPXTA |
| | A recognition sequence, with X | |
| | being any amino acid | |
| 58 | Staphylococcus aureus sortase | -NPQTN |
| | recognition sequence | |
| 59 | Linker derived from | -LPXT (Gn)- |
| | Staphylococcus aureus sortase | |
| | A recognition sequence, with X | |
| | being any amino acid and n | |
| | ≥1 and ≤21 | |
| 60 | Linker derived from | -LPXA (Gn)- |
| | Staphylococcus aureus sortase | |
| | A recognition sequence, with X | |
| | being any amino acid and n | |
| | ≥1 and ≤21 | |
| 61 | Linker derived from recognition | -LPXS (Gn)- |
| | sequence for Staphylococcus | |
| | aureus sortase A or engineered | |
| | sortase A 4S-9 from | |
| | Staphylococcus aureus, with X | |
| | being any amino acid and n | |
| | ≥1 and ≤21 | |
| 62 | Linker derived from recognition | -LAXT (Gn)- |
| | sequence for engineered sortase | |
| | A 2A-9 from Staphylococcus | |
| | aureus, with X being any amino | |
| | acid and n ≥1 and ≤21 | |
| 63 | Linker derived from | -LPXT (Gn)- or |
| | Streptococcus pyogenes sortase | -LPXT (An)- |
| | A recognition sequence, with X | |
| | being any amino acid and n ≥1 | |
| | and ≤21 | |
| 64 | Linker derived from | -NPQT (Gn)- |
| | Staphylococcus aureus sortase | |
| | recognition sequence, with | |
| | n ≥1 and ≤21 | |
| 65 | signal sequence | MNFGLRLIFLVLTLKGVQC |
| 66 | strepII-tag | WSHPQFEK |
| 67 | scFv-IgG_324_4-2 HC sequence | QSLEESGGGLVQPGESLTLTCT |
| | | VSGFSLSRNGMTWVRQAPGKGL |
| | | EWIGIITSSGDKYYATWAKGRF |
| | | TISKTSSTTVDLKMTSLTTEDT |
| | | ATYFCARGTVSSDIWGPGTLVT |
| | | ISSGGGGSGGGGSGGGGSGGGG |
| | | SELVLTQTPSPVSAAVGGTVTI |
| | | NCQASQSVYGNNELAWYQQKPG |
| | | QPPKLLIYRASILTSGVPSRFK |
| | | GSGSGTQFTLTISNVQREDAAT |
| | | YYCLGGYVSQSYRAAFGGGTEL |
| | | EILGGGSGGGSGGGSGGGSGGG |
| | | SQEQQKESGGGLFKPTDTLTLT |
| | | CTASGFDISSYYMSWVRQAPGN |
| | | GLEWIGAIGISGNAYYASWAKS |
| | | RSTITRNTNLNTVTLKMTSLTA |
| | | ADTATYFCARDHPTYGMDLWGP |
| | | GTLVTVSSASTKGPSVFPLAPS |
| | | SKSTSGGTAALGCLVKDYFPEP |
| | | VTVSWNSGALTSGVHTFPAVLQ |
| | | SSGLYSLSSVVTVPSSSLGTQT |
| | | YICNVNHKPSNTKVDKKVEPKS |
| | | CDKTHTCPPCPAPELLGGPSVF |
| | | LFPPKPKDTLMISRTPEVTCVV |
| | | VDVSHEDPEVKFNWYVDGVEVH |
| | | NAKTKPREEQYNSTYRVVSVLT |
| | | VLHQDWLNGKEYKCKVSNKALP |
| | | APIEKTISKAKGQPREPQVYTL |
| | | PPSRDELTKNQVSLTCLVKGFY |
| | | PSDIAVEWESNGQPENNYKTTP |
| | | PVLDSDGSFFLYSKLTVDKSRW |
| | | QQGNVFSCSVMHEALHNHYTQK |
| | | SLSLSPGR |
| 68 | scFv-IgG_324_4-2 LC sequence | SYELTQLPSVSVSLGQTARITC |
| | | EGNNIGSKAVHWYQQKPGLAPG |
| | | LLIYDDDERPSGVPDRFSGSNS |
| | | GDTATLTISGAQAGDEADYYCQ |
| | | VWDSSAYVFGGGTQLTVTGQPK |
| | | AAPSVTLFPPSSEELQANKATL |
| | | VCLISDFYPGAVTVAWKADSSP |
| | | VKAGVETTTPSKQSNNKYAASS |
| | | YLSLTPEQWKSHKSYSCQVTHE |
| | | GSTVEKTVAPTECS |
| 69 | ERR1-324 VL CDR1 | QASQSVYGNNELA |
| 70 | ERR1-324 VL CDR2 | RASILTS |
| 71 | ERR1-324 VL CDR3 | LGGYVSQSYRAA |
| 72 | ERR1-324 VH CDR1 | RNGMT |
| 73 | ERR1-324 VH CDR2 | IITSSGDKYYATWAKG |
| 74 | ERR1-324 VH CDR3 | GTVSSDI |
| 75 | 2A2 VH CDR1 | GYTFSDYEMH |
| 76 | 2A2 VH CDR2 | AIDPETGGTAYNQKFKG |
| 77 | 2A2 VH CDR3 | YYDYDSFTY |
| 78 | 2A2 VL CDR1 | KASQNVDAAVA |
| 79 | 2A2 VL CDR2 | SASNRYT |
| 80 | 2A2 VL CDR3 | QQYDIYPYT |
| 81 | XBR1-402 VH CDR1 | SYYMS |
| 82 | XBR1-402 VH CDR2 | AIGISGNAYYASWAKS |
| 83 | XBR1-402 VH CDR3 | DHPTYGMDL |
| 84 | XBR1-402 VL CDR1 | EGNNIGSKAVH |
| 85 | XBR1-402 VL CDR2 | DDDERPS |
| 86 | XBR1-402 VL CDR3 | QVWDSSAYV |
| 87 | R12 VH CDR1 | AYYMS |
| 88 | R12 VH CDR2 | TIYPSSGKTYYATWVNG |
| 89 | R12 VH CDR3 | DSYADDGALFNI |
| 90 | R12 VL CDR1 | TLSSAHKTDTID |
| 91 | R12 VL CDR2 | GSYTKRP |
| 92 | R12 VL CDR3 | GADYIGGYV |
| 93 | TOP43 VH CDR1 | SYWMS |
| 94 | TOP43 VH CDR2 | AIYGSGNTYYASWAKG |
| 95 | TOP43 VH CDR3 | DVHSTATDL |
| 96 | TOP43 VL CDR1 | GGNNIGSKAVN |
| 97 | TOP43 VL CDR2 | NDDERPS |
| 98 | TOP43 VL CDR3 | QLWDSSAGAYV |

What is claimed is:

1. A multi-specific product comprising
(a) a first entity comprising an antigen-binding domain that
binds to the same ROR1 epitope as and/or
competes for ROR1 binding with
antibody ERR1-324 comprising a heavy chain variable region sequence shown in SEQ ID NO. 2 and a light chain variable region sequence shown in SEQ ID NO. 3; and
(b) a second entity comprising an antigen-binding domain that binds to
to a different ROR1 epitope than, and/or
does not compete for binding with
antibody ERR1-324 comprising a heavy chain variable region sequence shown in SEQ ID NO. 2 and a light chain variable region sequence shown in SEQ ID NO. 3.

2. The product according to claim 1, which is a multi-specific antibody, alternative scaffold or antibody mimetic, wherein
(a) the first entity is a first antigen-binding domain that
binds to the same ROR1 epitope as and/or
competes for ROR1 binding with
antibody ERR1-324 comprising a heavy chain variable region sequence shown in SEQ ID NO. 2 and a light chain variable region sequence shown in SEQ ID NO. 3; and
(b) the second entity is a second antigen-binding domain that
binds to a different ROR1 epitope than, and/or
does not compete for binding with
antibody ERR1-324 comprising a heavy chain variable region sequence shown in SEQ ID NO. 2 and a light chain variable region sequence shown in SEQ ID NO. 3.

3. The product according to claim 1, which comprises two or more antibodies, alternative scaffolds or antibody mimetics, wherein
(a) the first entity is a first antibody comprising an antigen-binding domain that
binds to the same ROR1 epitope as and/or
competes for ROR1 binding with
antibody ERR1-324 comprising a heavy chain variable region sequence shown in SEQ ID NO. 2 and a light chain variable region sequence shown in SEQ ID NO. 3; and
(b) the second entity is a second antibody comprising an antigen-binding domain that
binds to a different ROR1 epitope than, and/or
does not compete for binding with
antibody ERR1-324 comprising a heavy chain variable region sequence shown in SEQ ID NO. 2 and a light chain variable region sequence shown in SEQ ID NO. 3.

4. The product according to any one of claims 1-3, wherein the entity comprising an antigen-binding domain is at least one selected from the group consisting of an antibody, an antibody-based binding protein, a bi-epitope-reactive antibody, a modified antibody format retaining target binding capacity, an antibody derivative or a fragment retaining target binding capacity, an alternative scaffold and/or an antibody mimetic.

5. The product according to any one of claims 1-4, wherein the antibody is at least one selected from the group consisting of an antibody, an antibody-based binding protein, a bi-epitope-reactive antibody, a modified antibody format retaining target binding capacity, an antibody derivative or a fragment retaining target binding capacity.

6. The product according to any one of claims 3-5, wherein the first and/or the second antibody, alternative scaffold or antibody mimetic is monospecific or mono-epitope reactive.

7. The product according to any one of claims 1-6, wherein the second antibody, alternative scaffold or antibody mimetic, or the second antigen-binding domain binds to ROR1, yet
to a different epitope than, and/or
does not compete for binding with
antibody ERR1-324 comprising a heavy chain variable region sequence shown in SEQ ID NO. 2 and a light chain variable region sequence shown in SEQ ID NO. 3.

8. The product according to claim 7, wherein the second antibody, alternative scaffold or antibody mimetic, or second antigen-binding domain
binds to the same ROR1 epitope as and/or
competes for ROR1 binding with
at least one antibody selected from the group consisting of
a) R12, comprising a heavy chain variable region sequence shown in SEQ ID NO. 20 and a light chain variable region sequence shown in SEQ ID NO. 21,
b) ERR1-TOP43, comprising a heavy chain variable region sequence shown in SEQ ID NO. 12 and a light chain variable region sequence shown in SEQ ID NO. 13,
c) ERR1-301, comprising a heavy chain variable region sequence shown in SEQ ID NO. 4 and a light chain variable region sequence shown in SEQ ID NO. 5,
d) ERR1-402, comprising a heavy chain variable region sequence shown in SEQ ID NO. 8 and a light chain variable region sequence shown in SEQ ID NO. 9, and/or
e) 2A2, comprising a heavy chain variable region sequence shown in SEQ ID NO. 22 and a light chain variable region sequence shown in SEQ ID NO. 23.

9. The product according to any one of claims 1-8, wherein ROR1 is human ROR1 (hROR1).

10. The product according to any one of claims 2 and 4-9, which is in a format selected from the group consisting of
bispecific or bi-epitope reactive scFv-Fc,
bispecific or bi-epitope reactive scFv-Ig, and/or
DVD-Ig.

11. The multi-specific product according to any one of claims 1-10, wherein the first entity comprises the following CDRs:
QASQSVYGNNELA (V_{L} CDR1, SEQ ID NO. 69),
RASILTS (V_{L} CDR2, SEQ ID NO. 70),
LGGYVSQSYRAA (V_{L} CDR3, SEQ ID NO. 71),
RNGMT (V_{H} CDR1 SEQ ID NO. 72),
IITSSGDKYYATWAKG (V_{H} CDR2, SEQ ID NO. 73), and
GTVSSDI (V_{H} CDR3, SEQ ID NO. 74),
and wherein the CDRs are comprised in a suitable protein framework so as to be capable to bind to ROR1.

12. The multi-specific product according to any one of claims 1-10, wherein the first entity comprises the heavy chain variable region sequence of antibody ERR1-324 shown in SEQ ID NO. 2 and the light chain variable region sequence of antibody ERR1-324 shown in SEQ ID NO. 3.

13. The multi-specific product according to any one of claims 1-10, wherein the second entity comprises at least one of the following sequence pairs:
a) the heavy chain variable region sequence of antibody R12 shown in SEQ ID NO. 20 and the light chain variable region sequence shown in SEQ ID NO. 21,
b) the heavy chain variable region sequence of antibody ERR1-TOP43 shown in SEQ ID NO. 12 and the light chain variable region sequence of antibody ERR1-TOP43 shown in SEQ ID NO. 13,
c) the heavy chain variable region sequence of antibody ERR1-301 shown in SEQ ID NO. 4 and the light chain variable region sequence of antibody ERR1-301 shown in SEQ ID NO. 5,
d) the heavy chain variable region sequence of antibody ERR1-402 shown in SEQ ID NO. 8 and the light chain variable region sequence of antibody ERR1-402 shown in SEQ ID NO. 9, and/or
e) the heavy chain variable region sequence of antibody 2A2 shown in SEQ ID NO. 22 and the light chain variable region sequence of antibody 2A2 shown in SEQ ID NO. 23.

14. An antibody drug conjugate (ADC) having the general formula A-(L)n-(T)m, in which
A is at least one antigen binding domain, antibody, alternative scaffold or antibody mimetic according to any one of claims 2-13,
L is a linker,
T is a toxin.
and in which n and m are integers between >1 and <10.

15. An antibody effector conjugate (AEC) having the general formula A-(L)n-(E)m, in which
A is at least one antigen binding domain, antibody, alternative scaffold or antibody mimetic according to any one of claims 2-13,
L is a linker,
E is a label
and in which n and m are integers between >1 and <10.

16. The conjugate according to any of claims 14-15, wherein the antibody is a multispecific antibody according to any of claims 2 and 4-13.

17. A composition comprising at least two antibody effector conjugates (AEC) or antibody drug conjugates (ADC) according to any one of claims 14-16, wherein each of the two conjugates comprises one of the monospecific antibodies, alternative scaffolds or antibody mimetics of any of claims 3-8.

18. The conjugate according to any one of claims 14-16, wherein the linker is at least one selected from the group consisting of
an oligopeptide linker
a maleimide linker, optionally comprising cleavable spacers, that may be cleaved by changes in pH, redox potential and or specific intracellular or extracellular enzymes.

19. The conjugate or composition according to any one of claims 14-18, wherein the linker has at least one of the following amino acid sequences: -LPXTGn-, -LPXAGn-, -LPXSGn-, -LAXTGn-, -LPXTGn-, -LPXTAn- or -NPQTGn-, with Gn being an oligo- or polyglycine or polyalanine with n being an integer between ≥1 and ≤21, and X being any conceivable amino acid sequence.

20. The conjugate or composition according to any one of claims 14-19, wherein the linker is conjugated to the C-terminus of at least one subdomain of the antibody.

21. The conjugate or composition according to any one of claims 14-20, wherein, prior to conjugation
the antibody bears a sortase recognition tag used or conjugated to the C-terminus of at least one subdomain thereof, and
the toxin or label comprises a glycine stretch with a length of between ≥1 and ≤20 glycine residues, preferably with a length of ≥2 and ≤5 glycine residues.

22. The conjugate or composition according to any one of claims 14-21, wherein, the toxin, or derivative thereof, is at least one selected from the group consisting of:
maytansinoids,
auristatins,
anthracyclins, preferably PNU-159682 derived anthracyclins
calicheamicins,
tubulysins
duocarmycins
radioisotopes
liposomes comprising a toxid payload
protein toxins
taxanes, and/or
pyrrolobenzodiazepines.

23. The conjugate or composition according to any one of claims 14-22, which is created by sortase-mediated conjugation of (i) an antibody carrying one or more sortase recognition tags and (ii) one or more toxins or labels carrying an oligoglycine tag.

24. A method of producing a conjugate according to any one of claims 14-23, which method comprises the following steps:
a) providing an antibody, alternative scaffold or antibody mimetic according to any one of claims 2-13, which antibody carries a sortase recognition tag,
b) providing one or more toxins or labels carrying an oligoglycine tag, and
c) conjugating the antibody and the toxin or label by means of sortase-mediated conjugation.

25. Use of the multispecific product according to any one of claims 1-13 or the antibody drug conjugate according to any one of claims 14 and 16-23, for the treatment of a patient that is
suffering from,
at risk of developing, and/or
being diagnosed for
a neoplastic disease.

26. A pharmaceutical composition comprising the multispecific product according to any one of claims 1-13 or the antibody drug conjugate according to any one of claims 14 and 16-23, together with one or more pharmaceutically acceptable ingredients.

27. A method of killing or inhibiting the growth of a cell expressing ROR1 in vitro or in a patient, which method comprises administering to the cell a pharmaceutically effective amount or dose of the multispecific product according to any one of claims 1-13, the antibody drug conjugate according to any one of claims 14 and 16-23, or of the pharmaceutical composition according to claim 26.

28. The method according to claim 27, wherein the cell expressing ROR1 is a cancer cell.


SEQUENCE LISTING (98 sequences)

SEQ ID NO: 1 - protein, 377 aa, Artificial Sequence
  >hROR1 extracellular domain amino acid sequence [organism=Artificial Sequence]
QETELSVSAELVPTSSWNISSELNKDSYLTLDEPMNNITTSLGQTAELHCKVSGNPPPTIRWFKNDAPVVQEPRRLSFRSTIYGSRLRIRNLDTTDTGYFQCVATNGKEVVSSTGVLFVKFGPPPTASPGYSDEYEEDGFCQPYRGIACARFIGNRTVYMESLHMQGEIENQITAAFTMIGTSSHLSDKCSQFAIPSLCHYAFPYCDETSSVPKPRDLCRDECEILENVLCQTEYIFARSNPMILMRLKLPNCEDLPQPESPEAANCIRIGIPMADPINKNHKCYNSTGVDYRGTVSVTKSGRQCQPWNSQYPHTHTFTALRFPELNGGHSYCRNPGNQKEAPWCFTLDENFKSDLCDIPACDSKDSKEKNKMEILY

SEQ ID NO: 2 - protein, 443 aa, Artificial Sequence
  >ERR1-324HC amino acid sequence [organism=Artificial Sequence]
QSLEESGGGLVQPGESLTLTCTVSGFSLSRNGMTWVRQAPGKGLEWIGIITSSGDKYYATWAKGRFTISKTSSTTVDLKMTSLTTEDTATYFCARGTVSSDIWGPGTLVTISSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 219 aa, Artificial Sequence
  >ERR1-324LC amino acid sequence [organism=Artificial Sequence]
ELVLTQTPSPVSAAVGGTVTINCQASQSVYGNNELAWYQQKPGQPPKLLIYRASILTSGVPSRFKGSGSGTQFTLTISNVQREDAATYYCLGGYVSQSYRAAFGGGTELEILRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 448 aa, Artificial Sequence
  >ERR1-301HC amino acid sequence [organism=Artificial Sequence]
QEQLEESGGGLVTPGGSLTLTCTASGFTISTYHMSWVRQAPGKGLEWIGSIYAGSGSTYYASWVNGRFTISSNTTQNTVSLQMNSLTVADTATYFCARDHPSYGMDLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 214 aa, Artificial Sequence
  >ERR1-301LC amino acid sequence [organism=Artificial Sequence]
SYELTQLPSVSVSLGQTARITCGGNSIGSKAVNWYQQKPGLAPGLLIYDDDERPSGVPDRFSASNSGDTATLTISGAQAEDEADYYCQLWDSSAVAYVFGGGTQLTVTGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 6 - protein, 455 aa, Artificial Sequence
  >ERR1-306HC amino acid sequence [organism=Artificial Sequence]
QEQLKESGGGLVQPGGSLKLSCKASGFDLSNYGVSWVRQAPGKGLEWIGYIDPTFDYTYYASWVNGRFSISRENTQNTVSLQINSLTPADTATYFCARWVYGVDDYGDGNWLDLWGQGTLVTISSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 217 aa, Artificial Sequence
  >ERR1-306LC amino acid sequence [organism=Artificial Sequence]
QFVLTQSPSVSAALGASAKLTCTLSSAHKTYTIDWYQQQQGEAPRYLMELKSDGSYTKGTGVPDRFSGSSSGADRYLIIPSVQADDEADYYCGTDYSGGYVFGGGTQLTVTGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 8 - protein, 447 aa, Artificial Sequence
  >XBR1-402 HC amino acid sequence [organism=Artificial Sequence]
QEQQKESGGGLFKPTDTLTLTCTASGFDISSYYMSWVRQAPGNGLEWIGAIGISGNAYYASWAKSRSTITRNTNLNTVTLKMTSLTAADTATYFCARDHPTYGMDLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 212 aa, Artificial Sequence
  >XBR1-402 LC amino acid sequence [organism=Artificial Sequence]
SYELTQLPSVSVSLGQTARITCEGNNIGSKAVHWYQQKPGLAPGLLIYDDDERPSGVPDRFSGSNSGDTATLTISGAQAGDEADYYCQVWDSSAYVFGGGTQLTVTGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 10 - protein, 446 aa, Artificial Sequence
  >ERR1-403HC amino acid sequence [organism=Artificial Sequence]
QEQLKESGRGLVQPGGSLKLSCKASGFDFSGWYMTWVRQAPGKGLEWIGTIGTTKGRTYYASWVNGRFTISSDNAQNTVDLQMNSLTAADRATYFCVRGSDYFDLWGPGTLVTISSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 214 aa, Artificial Sequence
  >ERR1-403LC amino acid sequence [organism=Artificial Sequence]
SYELTQLPSVSVSLGQTARITCGGNSIGSKAVNWYQQKPGLAPGLLIYDDDERPSGVPARFSGSNSGDTATLTISGAQAGDEADYYCQLWDSSAGAYVFGGGTQLTVTGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 12 - protein, 444 aa, Artificial Sequence
  >Top43HC amino acid sequence [organism=Artificial Sequence]
QSLEESGGRLVTPGTPLTLTCTVSGFSLSSYWMSWVRQAPGKGLEWIGAIYGSGNTYYASWAKGRFTISKTSTTVDLKITSPTTEDTATYFCARDVHSTATDLWGPGTLVTISSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 214 aa, Artificial Sequence
  >Top43LC amino acid sequence [organism=Artificial Sequence]
SYELTQLPSVSVSLGQTARITCGGNNIGSKAVNWYQQKPGLAPGLLIYNDDERPSGVPDRFSGSNSGDTATLTISGAQAGDEADYYCQLWDSSAGAYVFGGGTQLTVTGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 14 - protein, 470 aa, Artificial Sequence
  >XBR1-402 scFv HC & LC amino acid sequence [organism=Artificial Sequence]
SYELTQLPSVSVSLGQTARITCEGNNIGSKAVHWYQQKPGLAPGLLIYDDDERPSGVPDRFSGSNSGDTATLTISGAQAGDEADYYCQVWDSSAYVFGGGTQLTVTGGGGSGGGGSGGGGSQEQQKESGGGLFKPTDTLTLTCTASGFDISSYYMSWVRQAPGNGLEWIGAIGISGNAYYASWAKSRSTITRNTNLNTVTLKMTSLTAADTATYFCARDHPTYGMDLWGPGTLVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 472 aa, Artificial Sequence
  >ERR1-324-scFv HC & LC amino acid sequence [organism=Artificial Sequence]
ELVLTQTPSPVSAAVGGTVTINCQASQSVYGNNELAWYQQKPGQPPKLLIYRASILTSGVPSRFKGSGSGTQFTLTISNVQREDAATYYCLGGYVSQSYRAAFGGGTELEILGGGGSGGGGSGGGGSQSLEESGGGLVQPGESLTLTCTVSGFSLSRNGMTWVRQAPGKGLEWIGIITSSGDKYYATWAKGRFTISKTSSTTVDLKMTSLTTEDTATYFCARGTVSSDIWGPGTLVTISSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 470 aa, Artificial Sequence
  >scFv-402-Fc-KIH-p1 (Knob) [organism=Artificial Sequence]
SYELTQLPSVSVSLGQTARITCEGNNIGSKAVHWYQQKPGLAPGLLIYDDDERPSGVPDRFSGSNSGDTATLTISGAQAGDEADYYCQVWDSSAYVFGGGTQLTVTGGGGSGGGGSGGGGSQEQQKESGGGLFKPTDTLTLTCTASGFDISSYYMSWVRQAPGNGLEWIGAIGISGNAYYASWAKSRSTITRNTNLNTVTLKMTSLTAADTATYFCARDHPTYGMDLWGPGTLVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 17 - protein, 472 aa, Artificial Sequence
  >scFv-ERR1-324-Fc-KIH-p2 (Hole) [organism=Artificial Sequence]
ELVLTQTPSPVSAAVGGTVTINCQASQSVYGNNELAWYQQKPGQPPKLLIYRASILTSGVPSRFKGSGSGTQFTLTISNVQREDAATYYCLGGYVSQSYRAAFGGGTELEILGGGGSGGGGSGGGGSQSLEESGGGLVQPGESLTLTCTVSGFSLSRNGMTWVRQAPGKGLEWIGIITSSGDKYYATWAKGRFTISKTSSTTVDLKMTSLTTEDTATYFCARGTVSSDIWGPGTLVTISSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 577 aa, Artificial Sequence
  >324-LL-R12 HC amino acid sequence [organism=Artificial Sequence]
QSLEESGGGLVQPGESLTLTCTVSGFSLSRNGMTWVRQAPGKGLEWIGIITSSGDKYYATWAKGRFTISKTSSTTVDLKMTSLTTEDTATYFCARGTVSSDIWGPGTLVTISSASTKGPSVFPLAPQEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 342 aa, Artificial Sequence
  >324-LL-R12 LC amino acid sequence [organism=Artificial Sequence]
ELVLTQTPSPVSAAVGGTVTINCQASQSVYGNNELAWYQQKPGQPPKLLIYRASILTSGVPSRFKGSGSGTQFTLTISNVQREDAATYYCLGGYVSQSYRAAFGGGTELEILRTVAAPSVFIFPPELVLTQSPSVSAALGSPAKITCTLSSAHKTDTIDWYQQLQGEAPRYLMQVQSDGSYTKRPGVPDRFSGSSSGADRYLIIPSVQADDEADYYCGADYIGGYVFGGGTQLTVTGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 20 - protein, 451 aa, Artificial Sequence
  >R12 HC amino acid sequence [organism=Artificial Sequence]
QEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 217 aa, Artificial Sequence
  >R12 LC amino acid sequence [organism=Artificial Sequence]
ELVLTQSPSVSAALGSPAKITCTLSSAHKTDTIDWYQQLQGEAPRYLMQVQSDGSYTKRPGVPDRFSGSSSGADRYLIIPSVQADDEADYYCGADYIGGYVFGGGTQLTVTGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 22 - protein, 448 aa, Artificial Sequence
  >2A2 HC amino acid sequence [organism=Artificial Sequence]
QVQLQQSGAELVRPGASVTLSCKASGYTFSDYEMHWVIQTPVHGLEWIGAIDPETGGTAYNQKFKGKAILTADKSSSTAYMELRSLTSEDSAVYYCTGYYDYDSFTYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 214 aa, Artificial Sequence
  >2A2 LC amino acid sequence [organism=Artificial Sequence]
DIVMTQSQKIMSTTVGDRVSITCKASQNVDAAVAWYQQKPGQSPKLLIYSASNRYTGVPDRFTGSGSGTDFTLTISNMQSEDLADYFCQQYDIYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 447 aa, Artificial Sequence
  >Ac10 HC amino acid sequence [organism=Artificial Sequence]
QIQLQQSGPEVVKPGASVKISCKASGYTFTDYYITWVKQKPGQGLEWIGWIYPGSGNTKYNEKFKGKATLTVDTSSSTAFMQLSSLTSEDTAVYFCANYGNYWFAYWGQGTQVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 218 aa, Artificial Sequence
  >Ac10 LC amino acid sequence [organism=Artificial Sequence]
DIVLTQSPASLAVSLGQRATISCKASQSVDFDGDSYMNWYQQKPGQPPKVLIYAASNLESGIPARFSGSGSGTDFTLNIHPVEEEDAATYYCQQSNEDPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - DNA, 60 bp, Artificial Sequence
  >pCEP4-hROR1-F [organism=Artificial Sequence]
atcctgtttctcgtagctgctgcaactggagcacactccgcccggggcgccgccgcccag

SEQ ID NO: 27 - DNA, 60 bp, Artificial Sequence
  >pCEP4-hROR1- Avi-tag-R [organism=Artificial Sequence]
ccactcgatcttctgggcctcgaagatgtcgttcaggccctccatcttgttcttctcctt

SEQ ID NO: 28 - DNA, 60 bp, Artificial Sequence
  >pCEP4-signal-F-KpnI [organism=Artificial Sequence]
gctgggtaccggcgcgccaccatggactggacttggagaatcctgtttctcgtagctgct

SEQ ID NO: 29 - DNA, 60 bp, Artificial Sequence
  >pCEP4-6HIS-R-XhoI [organism=Artificial Sequence]
gccggcctcgagtcagtgatggtgatggtggtgctcgtgccactcgatcttctgggcctc

SEQ ID NO: 30 - DNA, 52 bp, Artificial Sequence
  >hROR1-His_R [organism=Artificial Sequence]
cggcctcgagtcagtgatggtgatggtggtgctccatcttgttcttctcctt

SEQ ID NO: 31 - DNA, 99 bp, Artificial Sequence
  >SP-hROR2_F [organism=Artificial Sequence]
gctgggtaccggcgcgccaccatggactggacttggagaatcctgtttctcgtagctgctgcaactggagcacactccgaagtggaggttctggatccg

SEQ ID NO: 32 - DNA, 52 bp, Artificial Sequence
  >hROR2-His_R [organism=Artificial Sequence]
cggcctcgagtcagtgatggtgatggtggtgccccatcttgctgctgtctcg

SEQ ID NO: 33 - DNA, 41 bp, Artificial Sequence
  >XBR1-402_VH_F [organism=Artificial Sequence]
gaggaggagctcactctcaggagcagcagaaggagtccggg

SEQ ID NO: 34 - DNA, 60 bp, Artificial Sequence
  >XBR1-402_VH_R [organism=Artificial Sequence]
cgatgggcccttggtggaggctgaagagacggtgacgagggtccctggcccccagaggtc

SEQ ID NO: 35 - DNA, 60 bp, Artificial Sequence
  >XBR1-402_?_F [organism=Artificial Sequence]
gagaagcttgttgctctggatctctggtgcctacgggtcctatgagctgacacagctgcc

SEQ ID NO: 36 - DNA, 21 bp, Artificial Sequence
  >LEAD-B [organism=Artificial Sequence]
ggccatggctggttgggcagc

SEQ ID NO: 37 - DNA, 60 bp, Artificial Sequence
  >KpnI_AscI-Signal [organism=Artificial Sequence]
ggtaccggcgcgccaccatggactggacttggagaatcctgtttctcgtagctgctgcaa

SEQ ID NO: 38 - DNA, 20 bp, Artificial Sequence
  >CH1-internal_overlap-R [organism=Artificial Sequence]
gccgctggtcagggctcctg

SEQ ID NO: 39 - DNA, 20 bp, Artificial Sequence
  >CH1-internal_overlap-F [organism=Artificial Sequence]
caggagccctgaccagcggc

SEQ ID NO: 40 - DNA, 30 bp, Artificial Sequence
  >HC-CH3-R-XhoI [organism=Artificial Sequence]
ggcctcgagtcatttacccggagacaggga

SEQ ID NO: 41 - DNA, 54 bp, Artificial Sequence
  >ERR1-324 HC-F [organism=Artificial Sequence]
tttctcgtagctgctgcaactggagcacactcccagtcgctggaggagtccggg

SEQ ID NO: 42 - DNA, 54 bp, Artificial Sequence
  >ERR1-TOP43 HC-F [organism=Artificial Sequence]
tttctcgtagctgctgcaactggagcacactcccagtcgttggaggagtccggg

SEQ ID NO: 43 - DNA, 54 bp, Artificial Sequence; identical to SEQ ID NO: 42
  >ERR1-TOP54 HC-F [organism=Artificial Sequence]

SEQ ID NO: 44 - DNA, 36 bp, Artificial Sequence
  >VH-CH1-R-EheI [organism=Artificial Sequence]
ggagggcgccagggggaagaccgatgggcccttggt

SEQ ID NO: 45 - DNA, 54 bp, Artificial Sequence
  >ERR1-TOP43 LC-F [organism=Artificial Sequence]
tttctcgtagctgctgcaactggagcacactcctcctatgagctgacacagctg

SEQ ID NO: 46 - DNA, 54 bp, Artificial Sequence; identical to SEQ ID NO: 45
  >ERR1-TOP54 LC-F [organism=Artificial Sequence]

SEQ ID NO: 47 - DNA, 54 bp, Artificial Sequence
  >ERR1-324 KC-F [organism=Artificial Sequence]
tttctcgtagctgctgcaactggagcacactccgagctcgtgctgacccagact

SEQ ID NO: 48 - DNA, 30 bp, Artificial Sequence
  >LC-R-XhoI [organism=Artificial Sequence]
ggcctcgagttatgaacattctgtaggggc

SEQ ID NO: 49 - DNA, 30 bp, Artificial Sequence
  >KC-R-XhoI [organism=Artificial Sequence]
ggcctcgagttaacactctcccctgttgaa

SEQ ID NO: 50 - DNA, 99 bp, Artificial Sequence
  >SP-hROR1_F [organism=Artificial Sequence]
gctgggtaccggcgcgccaccatggactggacttggagaatcctgtttctcgtagctgctgcaactggagcacactccgcccggggcgccgccgcccag

SEQ ID NO: 51 - protein, 450 aa, Artificial Sequence
  >Trastuzumab HC amino acid sequence [organism=Artificial Sequence]
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 214 aa, Artificial Sequence
  >Trastuzumab LC amino acid sequence [organism=Artificial Sequence]
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - protein, 5 aa, Artificial Sequence
  >Staphylococcus aureus sortase A recognition sequence, with X being any amino acid [organism=Artificial Sequence]
LPXTG

SEQ ID NO: 54 - protein, 5 aa, Artificial Sequence
  >Staphylococcus aureus sortase A recognition sequence, with X being any amino acid [organism=Artificial Sequence]
LPXAG

SEQ ID NO: 55 - protein, 5 aa, Artificial Sequence
  >recognition sequence for Staphylococcus aureus sortase A or engineered sortase A 4S-9 from Staphylococcus aureus, with X being any amino acid [organism=Artificial Sequence]
LPXSG

SEQ ID NO: 56 - protein, 5 aa, Artificial Sequence
  >recognition sequence for engineered sortase A 2A-9 from Staphylococcus aureus, with X being any amino acid [organism=Artificial Sequence]
LAXTG

SEQ ID NO: 57 - protein, 5 aa, Artificial Sequence
  >Streptococcus pyogenes sortase A recognition sequence, with X being any amino acid [organism=Artificial Sequence]
LPXTA

SEQ ID NO: 58 - protein, 5 aa, Artificial Sequence
  >Staphylococcus aureus sortase recognition sequence [organism=Artificial Sequence]
NPQTN

SEQ ID NO: 59 - protein, 5 aa, Artificial Sequence
  >Linker derived from Staphylococcus aureus sortase A recognition sequence, with X being any amino acid and n ? 1 and ? 21 [organism=Artificial Sequence]
LPXTG

SEQ ID NO: 60 - protein, 5 aa, Artificial Sequence
  >Linker derived from Staphylococcus aureus sortase A recognition sequence, with X being any amino acid and n ? 1 and ? 21 [organism=Artificial Sequence]
LPXAG

SEQ ID NO: 61 - protein, 5 aa, Artificial Sequence
  >Linker derived from recognition sequence for Staphylococcus aureus sortase A or engineered sortase A 4S-9 from Staphylococcus aureus, with X being any amino acid and n ? 1 and ? 21 [organism=Artificial Sequence]
LPXSG

SEQ ID NO: 62 - protein, 5 aa, Artificial Sequence
  >Linker derived from recognition sequence for engineered sortase A 2A-9 from Staphylococcus aureus, with X being any amino acid and n ? 1 and ? 21 [organism=Artificial Sequence]
LAXTG

SEQ ID NO: 63 - protein, 8 aa, Artificial Sequence
  >Linker derived from Streptococcus pyogenes sortase A recognition sequence, with X being any amino acid and n ? 1 and ? 21 [organism=Artificial Sequence]
LPXTLPXT

SEQ ID NO: 64 - protein, 5 aa, Artificial Sequence
  >Linker derived from Staphylococcus aureus sortase recognition sequence, with n ? 1 and ?21 [organism=Artificial Sequence]
NPQTG

SEQ ID NO: 65 - protein, 19 aa, Artificial Sequence
  >signal sequence [organism=Artificial Sequence]
MNFGLRLIFLVLTLKGVQC

SEQ ID NO: 66 - protein, 8 aa, Artificial Sequence
  >strepII-tag [organism=Artificial Sequence]
WSHPQFEK

SEQ ID NO: 67 - protein, 712 aa, Artificial Sequence
  >scFv-Ig_324_4-2 HC sequence [organism=Artificial Sequence]
QSLEESGGGLVQPGESLTLTCTVSGFSLSRNGMTWVRQAPGKGLEWIGIITSSGDKYYATWAKGRFTISKTSSTTVDLKMTSLTTEDTATYFCARGTVSSDIWGPGTLVTISSGGGGSGGGGSGGGGSGGGGSELVLTQTPSPVSAAVGGTVTINCQASQSVYGNNELAWYQQKPGQPPKLLIYRASILTSGVPSRFKGSGSGTQFTLTISNVQREDAATYYCLGGYVSQSYRAAFGGGTELEILGGGSGGGSGGGSGGGSGGGSQEQQKESGGGLFKPTDTLTLTCTASGFDISSYYMSWVRQAPGNGLEWIGAIGISGNAYYASWAKSRSTITRNTNLNTVTLKMTSLTAADTATYFCARDHPTYGMDLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGR

SEQ ID NO: 68 - protein, 212 aa, Artificial Sequence; identical to SEQ ID NO: 9
  >scFv-Ig_324_4-2 LC sequence [organism=Artificial Sequence]

SEQ ID NO: 69 - protein, 13 aa, Artificial Sequence
  >ERR1-324 VL CDR1 [organism=Artificial Sequence]
QASQSVYGNNELA

SEQ ID NO: 70 - protein, 7 aa, Artificial Sequence
  >ERR1-324 VL CDR2 [organism=Artificial Sequence]
RASILTS

SEQ ID NO: 71 - protein, 12 aa, Artificial Sequence
  >ERR1-324 VL CDR3 [organism=Artificial Sequence]
LGGYVSQSYRAA

SEQ ID NO: 72 - protein, 5 aa, Artificial Sequence
  >ERR1-324 VH CDR1 [organism=Artificial Sequence]
RNGMT

SEQ ID NO: 73 - protein, 16 aa, Artificial Sequence
  >ERR1-324 VH CDR2 [organism=Artificial Sequence]
IITSSGDKYYATWAKG

SEQ ID NO: 74 - protein, 7 aa, Artificial Sequence
  >ERR1-324 VH CDR3 [organism=Artificial Sequence]
GTVSSDI

SEQ ID NO: 75 - protein, 10 aa, Artificial Sequence
  >2A2 VH CDR1
GYTFSDYEMH

SEQ ID NO: 76 - protein, 17 aa, Artificial Sequence
  >2A2 VH CDR2
AIDPETGGTAYNQKFKG

SEQ ID NO: 77 - protein, 9 aa, Artificial Sequence
  >2A2 VH CDR3
YYDYDSFTY

SEQ ID NO: 78 - protein, 11 aa, Artificial Sequence
  >2A2 VL CDR1
KASQNVDAAVA

SEQ ID NO: 79 - protein, 7 aa, Artificial Sequence
  >2A2 VL CDR2
SASNRYT

SEQ ID NO: 80 - protein, 9 aa, Artificial Sequence
  >2A2 VL CDR3
QQYDIYPYT

SEQ ID NO: 81 - protein, 5 aa, Artificial Sequence
  >XBR1-402 VH CDR1
SYYMS

SEQ ID NO: 82 - protein, 16 aa, Artificial Sequence
  >XBR1-402 VH CDR2
AIGISGNAYYASWAKS

SEQ ID NO: 83 - protein, 9 aa, Artificial Sequence
  >XBR1-402 VH CDR3
DHPTYGMDL

SEQ ID NO: 84 - protein, 11 aa, Artificial Sequence
  >XBR1-402 VL CDR1
EGNNIGSKAVH

SEQ ID NO: 85 - protein, 7 aa, Artificial Sequence
  >XBR1-402 VL CDR2
DDDERPS

SEQ ID NO: 86 - protein, 9 aa, Artificial Sequence
  >XBR1-402 VL CDR3
QVWDSSAYV

SEQ ID NO: 87 - protein, 5 aa, Artificial Sequence
  >R12 VH CDR1
AYYMS

SEQ ID NO: 88 - protein, 17 aa, Artificial Sequence
  >R12 VH CDR2
TIYPSSGKTYYATWVNG

SEQ ID NO: 89 - protein, 12 aa, Artificial Sequence
  >R12 VH CDR3
DSYADDGALFNI

SEQ ID NO: 90 - protein, 12 aa, Artificial Sequence
  >R12 VL CDR1
TLSSAHKTDTID

SEQ ID NO: 91 - protein, 7 aa, Artificial Sequence
  >R12 VL CDR2
GSYTKRP

SEQ ID NO: 92 - protein, 9 aa, Artificial Sequence
  >R12 VL CDR3
GADYIGGYV

SEQ ID NO: 93 - protein, 5 aa, Artificial Sequence
  >TOP43 VH CDR1
SYWMS

SEQ ID NO: 94 - protein, 16 aa, Artificial Sequence
  >TOP43 VH CDR2
AIYGSGNTYYASWAKG

SEQ ID NO: 95 - protein, 9 aa, Artificial Sequence
  >TOP43 VH CDR3
DVHSTATDL

SEQ ID NO: 96 - protein, 11 aa, Artificial Sequence
  >TOP43 VL CDR1
GGNNIGSKAVN

SEQ ID NO: 97 - protein, 7 aa, Artificial Sequence
  >TOP43 VL CDR2
NDDERPS

SEQ ID NO: 98 - protein, 11 aa, Artificial Sequence
  >TOP43 VL CDR3
QLWDSSAGAYV